SEQUENCE LISTING (9 sequences)

SEQ ID NO: 1 - DNA, 20 bp, Artificial Sequence
  Synthetic
gctcgtcgcattcggcacgc

SEQ ID NO: 2 - DNA, 20 bp, Artificial Sequence
  Synthetic
atggtatnaagcttgccana

SEQ ID NO: 3 - DNA, 220 bp, Artificial Sequence
  Synthetic
gctcgtcgcattcggcacgccgacaatcatcacactaggtcgtgcgtgcgagaccgctgttaatacgggataaggcagtccgctggtctcggatagcgcgcttccgacaccttgtataggacgcagcgtgagaccgacgcactggacgactaggctatattcagtcgcgacgtgtccgcggacggctagatgcctataattgtgcctcctcacgcgccgt

SEQ ID NO: 4 - DNA, 220 bp, Artificial Sequence
  Synthetic
atggtatcaagcttgccacagccgaagcagacttaatcacgtcgagctctctactgcatagattagcgtacataggcccggaacccgggacaaggtgtcatcatagatgtcagcactgggtagtggcctgcagctatgtaaatcacgcttggtaagttgggtaattctgtacaggtcgcgataatcagcgggaatcaggcggcagaatctcgagtactag

SEQ ID NO: 5 - DNA, 20 bp, Artificial Sequence
  Synthetic
atggtatnaagcttgccana

SEQ ID NO: 6 - DNA, 20 bp, Artificial Sequence
  Synthetic
gctcgtcgcattcggcacgc

SEQ ID NO: 7 - DNA, 20 bp, Artificial Sequence
  Synthetic
gctcgtcgcattcggcacgc

SEQ ID NO: 8 - protein, 6 aa, Artificial Sequence
  Synthetic
GGGGGG

SEQ ID NO: 9 - DNA, 20 bp, Artificial Sequence
  Synthetic
gctcgtcgcattcggcacgc

(19) United States
(12) Patent Application Publication
(10) Pub. No.: US 2017/0153257 A1
(43) Pub. Date: Jun. 1, 2017

(54) METHODS OF ESTIMATING LOGD OF TAGGED COMBINATORIAL LIBRARY COMPOUNDS

(71) Applicant: DiCE Molecules SV, LLC, Redwood City, CA (US)

(72) Inventors: Phillip Patten, Portola Valley, CA (US); Pehr Harbury, Portola Valley, CA (US); Donald Schmidt, Walnut Creek, CA (US); Martin Linsell, San Mateo, CA (US); Kenneth Wlasichuk, Brisbane, CA (US); Robin Prince, San Francisco, CA (US); Timothy J. Church, San Mateo, CA (US); Wouter Van der Linden, Menlo Park, CA (US)

(21) Appl. No.: 15/289,946

(22) Filed: Oct. 10, 2016

Related U.S. Application Data
(60) Provisional application No. 62/239,824, filed on Oct. 9, 2015.

(51) Int. Cl. G01N 33/92; G01N 33/68; B01D 15/32; G01N 30/02; G01N 33/15; C12Q 1/68; C07H 21/04

(52) U.S. Cl. CPC G01N 33/92; C12Q 1/6876; G01N 33/6803; C07H 21/04; G01N 30/02; G01N 33/15; B01D 15/325; C12Q 2600/136; G01N 2030/027

(57) ABSTRACT

Disclosed herein are methods of estimating LogD of compounds comprising a ligand operatively linked to a recognition element. The methods generally involve contacting the compounds with a matrix, measuring the interaction of the compounds with the matrix and relating the interaction of the compounds with the matrix to LogD of the compounds. Also, described herein, are novel compounds which include a ligand operatively linked to a recognition element and linkers that connect the ligand to the recognition element.

This application claims priority under 35 U.S.C. §119 (e) from U.S. Provisional Application Ser. No. 62/239,824, filed Oct. 9, 2015 which is hereby incorporated by reference in its entirety.

### FIELD

Disclosed herein are methods of estimating LogD of compounds comprising a ligand operatively linked to a recognition element. The methods generally involve contacting the compounds with a matrix, measuring the interaction of the compounds with the matrix and relating the interaction of the compounds with the matrix to LogD of the compounds. Also described herein, are novel compounds which include a ligand operatively linked to a recognition element and linkers that connect the ligand to the recognition element.

### BACKGROUND

Combinatorial libraries, which were first developed over thirty years ago, now routinely identify novel, high affinity ligands for wide variety of biological targets (e.g., receptors, enzymes, nucleic acids, etc.) and hence are of increasing importance in drug discovery. Tagged combinatorial libraries, particularly libraries which use DNA as a tag to record the synthetic steps undergone by ligands operatively attached to the DNA, are of particular current interest. Advances in DNA sequencing, PCR technology and ligand assay development, provide methods to identify and select ligands operatively linked to DNA that bind to a biological target, from complex mixtures of ligands operatively linked to DNA (Harbury, et al., U.S. Pat. No. 7,479,472; Liu et al., U.S. Pat. No. 7,070,928; Liu et al., U.S. Pat. No. 7,223,545; Liu et al., U.S. Pat. No. 7,442,160; Liu et al., U.S. Pat. No. 7,491,160; Liu et al., U.S. Pat. No. 7,557,068; Liu et al., U.S. Pat. No. 7,771,935; Liu et al., U.S. Pat. No. 7,807,408; Liu et al., U.S. Pat. No. 7,998,904; Liu et al., U.S. Pat. No. 8,017,323; Liu et al., U.S. Pat. No. 8,183,178; Pedersen et al., U.S. Pat. No. 7,277,713; Pedersen et al., U.S. Pat. No. 7,413,854; Gouliev et al., U.S. Pat. No. 7,704,925; Franch et al., U.S. Pat. No. 7,915,201; Gouliev et al., U.S. Pat. No. 8,722,583; Freskgard et al., U.S. Patent Application No. 2006/0269920; Freskgard et al., U.S. Patent Application No. 2012/0028812; Hansen et al., U.S. Pat. No. 7,928,211; Hansen et al., U.S. Pat. No. 8,202,823; Hansen et al., U.S. Patent Application No. 2013/0005581; Hansen et al., U.S. Patent Application No. 2013/0288929; Neri et al., U.S. Pat. No. 8,642,514; Neri et al., U.S. Pat. No. 8,673,824; Neri et al., U.S. Patent Application No. 2014/01288290; Morgan et al., U.S. Pat. No. 7,972,992; Morgan et al., U.S. Pat. No. 7,935,658; Morgan et al., U.S. Patent Application No. 2011/0136697; Morgan et al., U.S. Pat. No. 7,972,994; Morgan et al., U.S. Pat. No. 7,989,395; Morgan et al., U.S. Pat. No. 8,410,028; Morgan et al., U.S. Pat. No. 8,598,089; Morgan et al., U.S. patent application Ser. No. 14/085,271; Wagner et al., U.S. Patent Application No. 2012/0053901; Keefe et al., U.S. Patent Application No. 2014/0315762; Dower et al., U.S. Pat. No. 6,140,493; Lerner et al., U.S. Pat. No. 6,060,596; Dower et al., U.S. Pat. No. 5,789,162; Lerner et al., U.S. Pat. No. 5,723,598; Dower et al.; U.S. Pat. No. 5,708,153; Dower et al., U.S. Pat. No. 5,639,603; and Lerner et al., U.S. Pat. No. 5,573,905).

However, most combinatorial libraries where ligands are operatively linked with tagging moieties are assayed for a single activity, i.e., binding affinity for a particular biological target. Frequently, other properties, such as, for example, bioavailability, stability under physiological conditions, toxicity and/or lipophilicity (important for absorption and distribution) are also of great significance in identifying suitable drug candidates.

The ability to passively diffuse across biological membranes, which is correlated strongly with lipophilicity, is essential for most drugs administered orally and for all drugs whose biological targets are intracellular. Lipophilicity is, therefore, one of the most important properties for a drug candidate. For example, compounds with an octanol-water coefficient (log D) of greater than five are generally undesirable candidates for further development. The ability to accurately estimate this parameter based on computational approaches is very limited and experimental measurement of this parameter remains essential for drug development.

Although, lipophilicity can be routinely measured for individual organic compounds, methods for estimating lipophilicity for ligands operatively linked to recognition elements in a complex mixture of similar ligands such as those provided by tagged combinatorial chemistry methods have not yet been developed. Accordingly, what is needed are methods for estimating lipophilicity of members of combinatorial libraries, where the ligands are operatively linked with recognition elements. Such methods will greatly assist in identifying compounds derived from combinatorial libraries with properties amenable to further optimization as drug candidates and accordingly, increase the efficiency of drug development.

### SUMMARY

Provided herein are compounds and methods which satisfy these and other needs. In a first aspect, a method of estimating LogD of one compound which includes a ligand operatively linked to a recognition element is provided. The method includes contacting the compound with a lipid matrix, separating the compound absorbed by the lipid matrix from the compound not absorbed by the lipid matrix, measuring the amount of the compound absorbed by the lipid matrix and/or the amounts of the compound not absorbed by the lipid matrix where measurement of the amount of the compound absorbed by the lipid matrix and/or the amounts of the compound not absorbed by the lipid matrix provide an estimation of LogD of the compound.

In a second aspect, a method of estimating LogD of two or more compounds which include a ligand operatively linked to a recognition element is provided. The method includes contacting the compounds with a lipid matrix, separating compounds absorbed by the lipid matrix from compounds not absorbed by the lipid matrix, measuring the amounts of compounds absorbed by the lipid matrix and/or the amounts of compounds not absorbed by the lipid matrix where measurement of the amounts of compounds absorbed by the lipid matrix and/or the amounts of the compounds not absorbed by the lipid matrix provide an estimation of LogD of the compounds.

In a third aspect, a method of estimating LogD of one compound which includes a ligand operatively linked to a recognition element is provided. The method comprises contacting the compound with a gel matrix, applying a voltage gradient to the gel matrix and measuring the R_{f} of the compound on the gel matrix wherein the R_{f} of the compound on the gel matrix provides an estimate of LogD of the compound.

In a fourth aspect, a method of estimating LogD of two or more compounds which include a ligand operatively linked to a recognition element is provided. The method includes the steps of contacting the compounds with a gel matrix, applying a voltage gradient to the gel matrix and measuring the R_{f} of the compounds on the gel matrix wherein the R_{f} of the compounds on the gel matrix provides an estimate of LogD of the compounds.

In a fifth aspect, a method of estimating LogD of one compound which include a ligand operatively linked to a recognition element is provided. The method includes the steps of contacting the compound with a chromatographic matrix and measuring the retention times of the compound on the chromatographic matrix wherein the measured retention times of the compound on the chromatographic matrix provides an estimate of LogD of the compound.

In a sixth aspect, a method of estimating LogD of two or more compounds which include a ligand operatively linked to a recognition element is provided. The method includes the steps of contacting the compounds with a chromatographic matrix and measuring the retention times of the compounds on the chromatographic matrix wherein the measured retention times of the compounds on the chromatographic matrix provides an estimate of LogD of the compounds.

In a seventh aspect, a double stranded DNA molecule comprising one oligonucleotide operatively linked to a variable first ligand at the 3′ terminus hybridized to a complementary oligonucleotide operatively linked to a constant second ligand at the adjacent 5′ terminus wherein the LogD of the second ligand is greater than 6 is provided.

In an eighth aspect, above molecule is used in a method of estimating LogD of two or more first ligands of double stranded DNA molecules. The method includes the steps of contacting the double stranded DNA molecules with a lipid matrix, separating the double stranded DNA molecules absorbed by the lipid matrix from double stranded DNA molecules not absorbed by the lipid matrix and measuring the amounts of double stranded DNA molecules absorbed by the lipid matrix and/or the amounts of double stranded DNA molecules not absorbed by the matrix wherein measurement of the amounts of double stranded DNA molecules absorbed by the matrix and/or the amounts of the double stranded DNA molecules not absorbed by the matrix provide an estimate of LogD of the first ligands of the double stranded DNA molecules.

In a ninth aspect, a compound of Formula (I):

RE-(X₁)—(C₁X₂)ₙ—(C₂X₃)ₒ-Lₐ (I)

or salts, hydrates, or solvates thereof is provided, where RE is a recognition element, Lₐ is a ligand, C₁ and C₂ are independently connecting elements, n and o are independently 0 or 1; and X₁, X₂ and X₃ are functional groups.

In still another aspect, a compound of Formula (II) is presented:

RE_{c}-(X₄)—(C₃X₅)ₖ—(C₄X₆)ₗL_{b} (II)

or salts, hydrates, or solvates thereof is provided where RE_{c} is a recognition element, L_{b} is a ligand with similar or identical hydrophobicity, C₃ and C₄ are independently linkers, X₄, X₅ and X₆ are functional groups and k and 1 or independently 0 or 1.

In an eleventh aspect, a compound comprising a compound of Formula (I) hybridized to a compound of Formula (II) wherein RE and RE_{c} are independently an oligonucleotide, single stranded RNA or single stranded DNA, C₁, C₂, n, o, X₁, X₂, X₃, Lₐ, L_{b} C₃, C₄, k, l, X₄, X₅ and X₆ are as defined above is provided.

### BRIEF DESCRIPTION OF THE FIGURES

FIG. 1 illustrates that compounds 1 and 2 attached to the 5′ end of DNA molecules are modestly retarded on a standard agarose gel.

FIG. 2 illustrates that compounds 1 and 2 attached to the 5′ end of DNA molecules are retarded on a standard agarose gel which includes 0.9% (w/v) synthetic vesicles.

FIG. 3 is a HPLC trace which illustrates that compounds 4-7 were resolved by reversed phase HPLC.

FIG. 4 illustrates the recovered area for compounds 2-7 after mixing with a matrix.

FIG. 5 illustrates the recovered area for compounds 8-11 after mixing with a matrix.

FIG. 6 illustrates the recovered area for compounds 13-17 after mixing with a matrix.

FIG. 7 illustrates the structure of the amine linker and pyrrolo dC.

FIG. 8 illustrates analytical data for an exemplary bile acid conjugate.

FIG. 9 is a HPLC trace which illustrates that compounds 19-24 were resolved by reversed phase HPLC.

FIG. 10 illustrates the retention time of bile acids conjugated to 220 base pair oligonucleotide plotted against cLogP of the bile acid methyl amides

FIG. 11 illustrates the retention time of the peptides of Example 12 plotted against their measured eLogD values.

FIG. 12 illustrates the retention time of the peptide oligomers of Example 13 plotted against eLogD values of the free peptides.

### DETAILED DESCRIPTION

### Definitions

Unless defined otherwise, all technical and scientific terms used herein have the same meaning as is commonly understood by one of ordinary skill in the art to which this invention belongs. In the event that a plurality of definitions for a term exists, those in this section prevail unless stated otherwise.

It must be noted that as used herein and in the appended claims, the singular forms “a”, “an” and “the” include plural referents unless the context clearly dictates otherwise. Thus, for example, reference to “a tag” includes a plurality of such tags and reference to “the compound” includes reference to one or more compounds and equivalents thereof known to those skilled in the art, and so forth.

It is further noted that the claims may be drafted to exclude optional element. As such, this statement is intended to serve as antecedent basis for use of such exclusive terminology as “solely”, “only” and the like in connection with the recitation of claim elements, or the use of a “negative” limitation.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values while still describing the particular solid form.

“Alkyl” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

“Alkanyl” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” by itself or as part of another substituent, refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkanyldiyl, alkenyldiyl and/or alkynyldiyl is used. In some embodiments, the alkyldiyl group is (C₁-C₂₀) alkyldiyl. In other embodiments, the alkyldiyl group is (C₁-C₁₀) alkyldiyl. In still other embodiments, the alkyldiyl group is (C₁-C₆) alkyldiyl. In some embodiments, saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkyleno, defined infra) are preferred.

“Alkyleno” by itself or as part of another substituent, refers to a straight-chain alkyldiyl group having two terminal monovalent radical centers derived by the removal of one hydrogen atom from each of the two terminal carbon atoms of straight-chain parent alkane, alkene or alkyne. Typical alkyleno groups include, but are not limited to, methano; ethylenos such as ethano, etheno, ethyno; propylenos such as propano, prop[1]eno, propa[1,2]dieno, prop[1] yno, etc.; butylenos such as butano, but[1]eno, but[2]eno, buta[1,3]dieno, but[1]yno, but[2]yno, but[1,3]diyno, etc.; and the like. Where specific levels of saturation are intended, the nomenclature alkano, alkeno and/or alkyno is used. In some embodiments, the alkyleno group is (C₁-C₂₀) alkyleno. In other embodiments, the alkyleno group is (C₁-C₁₀) alkyleno. In still other embodiments, the alkyleno group is (C₁-C₆) alkyleno. In some embodiments, straight-chain saturated alkano groups, e.g., methano, ethano, propano, butano, and the like are preferred.

“Antibody” as used herein refers to a protein comprising one or more polypeptides substantially or partially encoded by immunoglobulin genes or fragments of immunoglobulin genes, e.g., a fragment containing one or more complementarity determining region (CDR). The recognized immunoglobulin genes include the kappa, lambda, alpha, gamma, delta, epsilon and mu constant region genes, as well as myriad immunoglobulin variable region genes. Light chains are typically classified as either, e.g., kappa or lambda. Heavy chains are typically classified e.g., as gamma, mu, alpha, delta, or epsilon, which in turn define the immunoglobulin classes, IgG, IgM, IgA, IgD and IgE, respectively. A typical immunoglobulin (antibody) structural unit comprises a tetramer. In nature, each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. Thus, for example, pepsin digests an antibody below the disulfide linkages in the hinge region to produce F(ab)′2 (fragment antigen binding) and Fc (fragment crystallizable, or fragment complement binding). F(ab)′2 is a dimer of Fab, which itself is a light chain joined to VH-CH₁ by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region. The Fc portion of the antibody molecule corresponds largely to the constant region of the immunoglobulin heavy chain, and is responsible for the antibody's effector function (see, Fundamental immunology, 4th edition. W. E. Paul, ed., Raven Press, N.Y. (1998), for a more detailed description of antibody fragments). While various antibody fragments are defined in terms of the digestion of an intact antibody, one of skill will appreciate that such Fab′ or Fc fragments may be synthesized de novo either chemically or by utilizing recombinant DNA methodology, peptide display, or the like. Thus, the term antibody, as used herein, also includes antibody fragments either produced by the modification of whole antibodies or synthesized de novo using recombinant DNA methodologies. Antibodies also include single-armed composite monoclonal antibodies, single chain antibodies, including single chain Fv (sFv) antibodies in which a variable heavy and a variable light chain are joined together (directly or through a peptide linker) to form a continuous polypeptide, as well as diabodies, tribodies, and tetrabodies (Pack et al. (1995) J Mol Biol 246:28; Biotechnol 1271; and Biochemistry 31:1579). The antibodies are, e.g., polyclonal, monoclonal, chimeric, humanized, single chain, Rib fragments, fragments produced by an Fab expression library, or the like,

“Aryl” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀ aryl).

“Arylalkyl” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl.

“Aryldiyl” by itself or as part of another substituent refers to a divalent hydrocarbon radical derived by the removal of one hydrogen atom from each of two different carbon atoms of a parent aromatic system or by removal of two hydrogen atoms from a single carbon atom of a parent aromatic ring system. The two monovalent radical centers or each valency of the divalent center can form bonds with the same or different atom(s). Typical aryldiyl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryldiyl group comprises from 5 to 20 carbon atoms. In other embodiments, an aryldiyl group comprises from 5 to 12 carbon atoms.

“Arylalkyldiyl” refers to an acyclic alkyl diradical in which one of the hydrogen atoms bonded to a carbon atom, typically a terminal or sp³ carbon atom, is replaced with an aryl group.

“cLogD,” as used herein, refers to a calculated value of LogD using the Chem Axon Physiochemical LogD/logP program. Note the two phases are not in equilibrium.

cLogP as used herein refers to a computed partition coefficient of a compound between water and a lipophilic phase (usually octanol) using the Chem Axon Physiochemical LogD/LogP Program. Note the two phases are not in equilibrium.

“Chromatographic matrix” as used herein, refers to conventional chromatography resins, such as, for example, reverse phase, silica, ion exchange or “mixed mode” supports. The chromatographic matrix may also include resins which include immobilized proteins (e.g., serum albumin, alpha-acid glycoprotein or immobilized artificial membranes (many are available from Regis Technologies, Inc., Morton Grove, Ill.) such as, for example, phospholipids (Pidgeon et al., Anal. Chem. 176 (1989)).

“ChromLogD,” as used herein, refers to a determination of LogD by chromatography. The chromLogD of an unknown compound is calculated by interpolation based of the retention time of two compounds of known eLogD that bracket the unknown compound and for which the retention time has been measured using the same HPLC method (ideally as a co-injection with the unknown compound).

“ChromₛₛLogD,” as used herein, refers to determination of a surrogate of eLogD of a compound by chromatography wherein the compound is covalently attached to a single stranded nucleic acid fragment, typically by an amide bond to a TEG linker. The retention time serves as the surrogate for eLogD of the compound when not attached to the single stranded nucleic acid. The chromLogD value of an unknown compound is calculated by interpolation based on the retention time of two compounds of known chromLogD that bracket the unknown compound and for which the retention time has been measured using the same HPLC method (ideally as a co-injection with the unknown compound) and an identical or functionally similar linker. In some embodiments, the nucleic acid is 20 base pair DNA oligonucleotide and the surrogate of eLogD is chromₛₛ₂₀LogD.

“Chrom_{ds}LogD,” as used herein refers to determination of a surrogate of eLogD that is analogous to chromₛₛLogD, with the key difference being that the conjugates are covalently attached to double stranded DNA fragment. In some embodiments, the nucleic acid is 220 base pair DNA oligonucleotide, which is similar in design to a typical library gene used in Harbury et al., U.S. Pat. No. 7,479,472 and the surrogate of eLogD is chrom_{ds220}LogD.

“Coding template” as used herein mean nucleic acid sequences which each comprise a plurality of hybridization sequences (i.e., codons) and a functional group or a linking entity. The “hybridization sequences” refer to oligonucleotides comprising between about 3 and up to 100, 3 and up to 50, and from about 5 to about 30 nucleic acid subunits. Such coding templates are capable of directing the synthesis of the combinatorial library based on the catenated hybridization sequences. The coding template is operatively linked to a functional group or optionally a linking entity. Coding templates may be immobilized by capture templates and direct combinatorial library synthesis in DPCC. In some embodiments, coding templates are oligonucleotides. In some embodiments, the hybridization sequences are 20 nucleic acid subunits. In other embodiments, hybridization sequences are separated by constant spacer sequences. Constant spacer sequences refer to oligonucleotides comprising between about 3 and up to 100, 3 and up to 50, and from about 5 to about 30 nucleic acid subunits.” refer to oligonucleotides comprising between about 3 and up to 100, 3 and up to 50, and from about 5 to about 30 nucleic acid subunits. In some embodiments, the constant spacer sequences are 20 nucleic acid subunits.

“Combinatorial library” as used herein refers to a library of molecules containing a large number, typically between 10³ and 10¹⁵ or more different compounds typically characterized by different sequences of subunits, or a combination of different side chains functional groups and linkages. In some embodiments, a combinatorial library includes more than 10² molecules.

“Compounds” refers to compounds encompassed by structural formulae disclosed herein and includes any specific compounds within these formulae whose structure is disclosed herein. Compounds may be identified either by their chemical structure and/or chemical name. When the chemical structure and chemical name conflict, the chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds described herein include, but are not limited to, ²H, ³H, ¹³C, ¹⁴C, ¹⁵N, ¹⁸O, ¹⁷O, ³⁵S, etc. In general, it should be understood that all isotopes of any of the elements comprising the compounds described herein may be found in these compounds. Compounds may exist in unsolvated or unhydrated forms as well as solvated forms, including hydrated forms and as N-oxides. In general, compounds may be hydrated, solvated or N-oxides. Certain compounds may exist in multiple crystalline or amorphous forms. In general, all physical forms are equivalent for the uses contemplated herein and are intended to be within the scope of the present invention. Further, it should be understood, when partial structures of the compounds are illustrated, that brackets indicate the point of attachment of the partial structure to the rest of the molecule.

“eLogD,” as used herein, refers to a determination of LogD by shake flask method in aqueous buffer/octanol mixture.

“Depsipeptide” as used herein refers to a peptide as defined herein where one or more of amide bonds are replaced by ester bonds.

“Gel matrix” as used herein includes, refers to various gels such as, cyrogels, agarose, superagarose or polyacrylamide gels. Typically, a gel matrix will include a lipid phase, such as, for example, vesicles, liposomes, micelles, lipophilic compounds, lipophilic polymers, artificial membranes or combinations thereof.

“Heteroalkyl, Heteroalkanyl, Heteroalkenyl, Heteroalkanyl, Heteroalkyldiyl and Heteroalkyleno” by themselves or as part of another substituent, refer to alkyl, alkanyl, alkenyl, alkynyl, alkyldiyl and alkyleno groups (and optionally any associated hydrogen atoms) are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰²—, ═N—N═, —N═N—, —N═N—NR⁵⁰³R⁵⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸— and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl” by itself or as part of another substituent refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylakenyl and/or heteroarylalkynyl is used. In some embodiments, the heteroarylalkyl group is a 6-21 membered heteroarylalkyl, e.g., the alkanyl, alkenyl or alkynyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the alkanyl, alkenyl or alkynyl moiety is (C₁-C₃) alkyl and the heteroaryl moiety is a 5-10 membered heteroaryl.

“Heteroaryldiyl” refers to a divalent heteroaromatic group derived by the removal of one hydrogen atom from each of two different atoms of a parent heteroaromatic ring system or by the removal of two hydrogen atoms from a single atom of a parent heteroaromatic ring system. The two monovalent radical centers or each valency of the single divalent center can form bonds with the same or different atom(s). Typical heteroaryldiyl groups include, but are not limited to, divalent groups derived from acridine, arsindole, carbazole, □-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryldiyl group is 5-20 membered heteroaryldiyl. In other embodiments the heteroaryldiyl group is 5-10 membered heteroaryldiyl. In some embodiments, heteroaryldiyl groups derived from thiophene, pyrrole, benzothiophene, benzofuran, indole, pyridine, quinoline, imidazole, oxazole and pyrazine are preferred.

“Heteroarylalkyldiyl” refers to an acyclic alkyl diradical in which one of the hydrogen atoms bonded to a carbon atom, typically a terminal or sp³ carbon atom, is replaced with an heteroaryl group.

“Hydrates” refers to incorporation of water into to the crystal lattice of a compound described herein, in stoichiometric proportions, resulting in the formation of an adduct. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. See, e.g., (Guillory, K., Chapter 5, pp. 202-205 in Polymorphism in Pharmaceutical Solids, (Brittain, H. ed.), Marcel Dekker, Inc., New York, N.Y., 1999). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-Ray diffraction, X-Ray powder diffraction, Polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of hydrates such as, for example, HOLODIAG, Pharmaparc II, Voie de l'Innovation, 27 100 Val de Reuil, France (http://www.holodiag.com).

“Lipid matrix” as used herein refers to lipophilic vesicles, artificial membranes, beads coated with lipophilic or amphophilic material, lipophilic compounds, lipophilic polymers, liposomes or micelles.

“LogD(pH),” as used herein, refers to a distribution of a compound between aqueous buffer and octanol at a given pH. Unless otherwise stated, the pH is assumed to be 7.4 and be written as LogD.

“Matrix” as used herein refers to, in general, at least three different types of matrixes: “chromatographic,” “gel,” or “lipid.” In some embodiments, the matrix may be a hydrophilic liquid contacting a hydrophobic liquid (e.g., octanol-water).

“Nucleic acid” as used herein refers to an oligonucleotide analog as defined below as well as a double stranded. DNA and RNA molecule. A DNA and RNA molecule may include the various analogs defined below.

“Oligonucleotides” or “oligos” as used herein refer to nucleic acid oligomers containing between about 3 and up to about 300, and typically from about 5 to about 300 nucleic acid subunits. In the context of oligos hybridization sequence) which direct the synthesis of library compounds, the oligos may include or be composed of naturally-occurring nucleotide residues, nucleotide analog residues, or other subunits capable of forming sequence-specific base pairing, when assembled in a linear polymer, with the proviso that the polymer is capable of providing a suitable substrate for strand-directed polymerization in the presence of a polymerase and one or more nucleotide triphosphates, e.g., conventional deoxyribonucleotides. A “known-sequence oligo” is an oligo whose nucleic acid sequence is known.

“Oligonucleotide analog” as used herein refers to a nucleic acid that has been modified and which is capable of some or all of the chemical or, biological activities of the oligonucleotide from which it was derived. An oligonucleotide analog will generally contain phosphodiester bonds, although in some cases, oligonucleotide analogs are included that may have alternate backbones. Modifications of the ribose-phosphate backbone may facilitate the addition of additional moieties such as labels, or may be done to increase the stability and half-life of such molecules. In addition, mixtures of naturally occurring nucleic acids and analogs can be made. Alternatively, mixtures of different nucleic acid analogs, and mixtures of naturally occurring nucleic acids and analogs may be made. The oligonucleotides may be single stranded or double stranded, as specified, or contain portions of both double stranded or single stranded sequence. The oligonucleotide may be DNA, RNA or a hybrid, where the nucleic acid contains any combination of deoxyribo-and ribo-nucleotides, and any combination of bases, including uracil, uridine, adenine, thymine, cytosine, guanine, inosine, xathanine hypoxathanine, isocytosine, isoguanine, etc.

“Operatively linked,” as used herein, means at least two chemical structures joined together in such a way as to remain linked through the various manipulations described herein. Typically, a ligand or functional group and the coding nucleotide are linked covalently via an appropriate linker. The linker is at least a bivalent moiety with a site of attachment for the oligonucleotide and a site of attachment for the ligand or a functional group. For example, when the functional moiety is a polyimide compound, the polyimide compound can be attached to the linking group at the N-terminus, the C-terminus or via a functional group on one of the side chains. The linker is sufficient to separate the ligand and the oligonucleotide by at least one atom and in some embodiments by more than one atom. In most embodiments, the linker is sufficiently flexible to allow the ligand to bind target molecules in a manner which is independent of the oligonucleotide.

“Peptide” as used herein refers to a polymer of amino acid residues between about 2 and 50 amino acid residues, between about 2 and 20 amino acid residues, or between about 2 and 10 residues. Peptides include modified peptides such as, for example, glycopeptides, PEGylated peptides, lipopeptides, peptides conjugated with organic or inorganic ligands, peptides which contain peptide bond isosteres (e.g., ψ[CH₂S], ψ[CH₂NH₂], ψ[NHCO], ψ[COCH₂], ψ[(E) or (Z) CH═CH], etc and also include cyclic peptides. In some embodiments, the amino acid residues may be any L-α-amino acid, D-α-amino residue, N-alkyl variants thereof or combinations thereof. In other embodiments, the amino acid residues may any L-α-amino acid, D-α-amino residue, β-amino acids, γ-amino acids, N-alkyl variants thereof or combinations thereof.

“Parent Aromatic Ring System” refers to an unsaturated cyclic or polycyclic ring system having a conjugated π electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Peptide nucleic acid” as used herein refers to oligonucleotide analogues where the sugar phosphate backbone of nucleic acids has been replaced by psuedopeptide skeleton (e.g., N-(2-aminoethyl)-glycine) (Nielsen et al., U.S. Pat. No. 5,539,082; Nielsen et al., U.S. Pat. No. 5,773,571; Burchardt et al., U.S. Pat. No. 6,395,474).

“Peptoid” as used hereinrefers to polymers of poly N-substituted glycine (Simon et al., Proc. Natl. Acad. Sci. (1992) 89(20) 9367-9371) and include cyclic variants thereof.

“Polypeptide” as used herein refers to a polymer of amino acid residues typically comprising greater than 50 amino acid residues and includes cyclic variants thereof. Polypeptide includes proteins (including modified proteins such as glycoproteins, PEGylated proteins, lipoproteins, polypeptide conjugates with organic or inorganic ligands, etc.) receptor, receptor fragments, enzymes, structural proteins (e.g., collagen) etc. In some embodiments, the amino acid residues may be any L-α-amino acid, D-α-amino residue, or combinations thereof. In other embodiments, the amino acid residues may be any L-α-amino acid, D-α-amino residue, N-alkyl variants thereof or combinations thereof.

“Recognition Element” as used herein refers to an oligonucleotide, single or double-stranded RNA, single or double-stranded DNA, a DNA binding protein, a locked nucleic acid, a RNA binding protein, a peptide nucleic acid, a peptide, a depsipeptide, a polypeptide, an antibody, a peptoid, a polymer, a polysiloxanes, an inorganic compounds of molecular weight greater that 50 daltons, organic compounds of molecular weight between about 2000 daltons and about 50 daltons or a combination thereof.

“Salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. In some embodiments, salts may be formed when an acidic proton present can react with inorganic bases (e.g., sodium hydroxide, sodium carbonate, potassium hydroxide, aluminum hydroxide, calcium hydroxide, etc.) and organic bases (e.g., ethanolamine, diethanolamine, triethanolamine, tromethamine, N-methylglucamine, etc.). In some embodiments, the salt is pharmaceutically acceptable.

“Solvates” refers to incorporation of solvents into to the crystal lattice of a compound described herein, in stoichiometric proportions, resulting in the formation of an adduct. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. See, e.g., (Guillory, K., Chapter 5, pp. 205-208 in Polymorphism in Pharmaceutical Solids, (Brittain, H. ed.), Marcel Dekker, Inc., New York, N.Y., 1999)). The above methods for preparing solvates are well within the ambit of those of skill in the art, are completely conventional do not require any experimentation beyond what is typical in the art. Solvates may be characterized and/or analyzed by methods well known to those of skill in the art such as, for example, single crystal X-Ray diffraction, X-Ray powder diffraction, Polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, 1999). In addition, many commercial companies routinely offer services that include preparation and/or characterization of solvates such as, for example, HOLODIAG, Pharmaparc II, Voie de l'Innovation, 27 100 Val de Reuil, France (http://www.holodiag.com).

“Standard compound” as used herein refers to, a compound of measured LogD, which also has a measured retention time in a chromatographic matrix, or a measured R_{f} in a gel matrix or a known absorbance in a lipid matrix both as a free compound and also when conjugated to a standard oligonucleotide (e.g., an oligonucleotide of 20 nucleic acid subunits or 220 nucleic acid subunits) has also measured. Standard compounds are co-injected with combinatorial library members, for example, on a chromatographic matrix. Retention time of the library members can then be compared with retention time of standard compounds to provide an estimate of LogD of library members.

“Substituted” when used to modify a specified group or radical, means that one or more hydrogen atoms of the specified group or radical are each, independently of one another, replaced with the same or different substituent(s). Substituent groups useful for substituting saturated carbon atoms in the specified group or radical include, but are not limited to —R^{a}, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, ═N₂, —N₃, —S(O)₂R^{b}, —S(O)₂NR^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a} is selected from the group consisting of alkyl, cycloalkyl, heteroalkyl, cycloheteroalkyl, aryl, arylalkyl, heteroaryl and heteroarylalkyl; each R^{b} is independently hydrogen or R^{a}; and each R^{c} is independently R^{b} or alternatively, the two R^{c}'s are taken together with the nitrogen atom to which they are bonded form a 4-, 5-, 6- or 7-membered cycloheteroalkyl which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl.

Similarly, substituent groups useful for substituting unsaturated carbon atoms in the specified group or radical include, but are not limited to, —R^{a}, halo, —O⁻, —OR^{b}, —SR^{b}, —S⁻, —NR^{c}R^{c}, trihalomethyl, —CF₃, —CN, —OCN, —SCN, —NO, —NO₂, —N₃, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} and R^{c} are as previously defined.

Substituent groups from the above lists useful for substituting other specified groups or atoms will be apparent to those of skill in the art. The substituents used to substitute a specified group can be further substituted, typically with one or more of the same or different groups selected from the various groups specified above. Combinations of substituents envisioned herein are those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein. In some embodiments, substituents are limited to the groups described explicitly above.

Reference will now be made in detail to embodiments of the invention. While the invention will be described in conjunction with these embodiments, it will be understood that it is not intended to limit the invention to the embodiments, infra. To the contrary, it is intended to cover alternatives, modifications, and equivalents as may be included within the spirit and scope of the invention as defined by the appended claims.

### Methods of Estimating LogD of Tagged Combinatorial Library Compounds

Described herein are methods for estimating LogD of compounds which include a ligand operatively linked to a recognition element. In some instances, the compounds may be members of combinatorial libraries and the methods may simultaneously provide estimates of LogD of a number of members of the combinatorial libraries.

Combinatorial libraries are well known and may be synthesized by methods known in the art (Harbury, et al., U.S. Pat. No. 7,479,472; Liu et al., U.S. Pat. No. 7,070,928; Liu et al., U.S. Pat. No. 7,223,545; Liu et al., U.S. Pat. No. 7,442,160; Liu et al., U.S. Pat. No. 7,491,160; Liu et al., U.S. Pat. No. 7,557,068; Liu et al., U.S. Pat. No. 7,771,935; Liu et al., U.S. Pat. No. 7,807,408; Liu et al., U.S. Pat. No. 7,998,904; Liu et al., U.S. Pat. No. 8,017,323; Liu et al., U.S. Pat. No. 8,183,178; Pedersen et al., U.S. Pat. No. 7,277,713; Pedersen et al., U.S. Pat. No. 7,413,854; Gouliev et al., U.S. Pat. No. 7,704,925; Franch et al., U.S. Pat. No. 7,915,201; Gouliev et al., U.S. Pat. No. 8,722,583; Freskgard et al., U.S. Patent Application No. 2006/0269920; Freskgard et al., U.S. Patent Application No. 2012/0028812; Hansen et al., U.S. Pat. No. 7,928,211; Hansen et al., U.S. Pat. No. 8,202,823; Hansen et al., U.S. Patent Application No. 2013/0005581; Hansen et al., U.S. Patent Application No. 2013/0288929; Neri et al., U.S. Pat. No. 8,642,514; Neri et al., U.S. Pat. No. 8,673,824; Neri et al., U.S. Patent Application No. 2014/01288290; Morgan et al., U.S. Pat. No. 7,972,992; Morgan et al., U.S. Pat. No. 7,935,658; Morgan et al., U.S. Patent Application No. 2011/0136697; Morgan et al., U.S. Pat. No. 7,972,994; Morgan et al., U.S. Pat. No. 7,989,395; Morgan et al., U.S. Pat. No. 8,410,028; Morgan et al., U.S. Pat. No. 8,598,089; Morgan et al., U.S. patent application Ser. No. 14/085,271; Wagner et al., U.S. Patent Application No. 2012/0053901; Keefe et al., U.S. Patent Application No. 2014/0315762; Dower et al., U.S. Pat. No. 6,140,493; Lerner et al., U.S. Pat. No. 6,060,596; Dower et al., U.S. Pat. No. 5,789,162; Lerner et al., U.S. Pat. No. 5,723,598; Dower et al.; U.S. Pat. No. 5,708,153; Dower et al., U.S. Pat. No. 5,639,603; and Lerner et al., U.S. Pat. No. 5,573,905).

Without wishing to be bound by theory, the structure of the ligand may determine the relative LogD of members of combinatorial libraries. The recognition elements (i.e., tags) are typically isomeric polymers and thus possess similar physiochemical properties. Accordingly, ligands of different polarity may control the LogD of compounds, which include ligands operatively linked to recognition elements (Bouma et al., U.S. Pat. No. 5,006,473, Pascoe et al., Electrophoresis 2003, 24, 4227-4240; Pascoe et al., Electrophoresis 2006, 27, 793-804). LogD of compounds which include a ligand operatively linked to a recognition element in combinatorial libraries may be estimated without modification or after binding of the recognition element to either a complementary moiety or a modified complementary moiety, infra.

In some embodiments, a method of estimating LogD of one compound which includes a ligand operatively linked to a recognition element is provided. The method includes contacting the compound with a lipid matrix, separating the compound absorbed by the lipid matrix from the compound not absorbed by the lipid matrix, measuring the amount of the compound absorbed by the lipid matrix and/or the amounts of the compound not absorbed by the lipid matrix where measurement of the amount of the compound absorbed by the lipid matrix and/or the amounts of the compound not absorbed by the lipid matrix provide an estimation of LogD of the compound.

In other embodiments, a method of estimating LogD of two or more compounds which include a ligand operatively linked to a recognition element is provided. The method includes contacting the compounds with a lipid matrix, separating compounds absorbed by the lipid matrix from compounds not absorbed by the lipid matrix, measuring the amounts of compounds absorbed by the lipid matrix and/or the amounts of compounds not absorbed by the lipid matrix where measurement of the amounts of compounds absorbed by the lipid matrix and/or the amounts of the compounds not absorbed by the lipid matrix provide an estimation of LogD of the compounds.

In some embodiments, an excipient is included in the contacting step. The excipient may be a cation or polycation such as, for example. a polyamine or a salt or a volume excluding agent such as PEG. The polyamine may be, but is not limited to, putrescine, cadaverine, spermidine or spermine. In some embodiments, the salt is a fluoride, sulfate, phosphate, acetate, chloride, nitrate, bromide, chloride, perchlorate or thiosulfate anion combined with ammonium, potassium, sodium, lithium, magnesium, calcium or guanadinium cation. In other embodiments, the salt is ammonium sulfate.

In some embodiments, the mixture is contacted with the matrix at a temperature between about 15° C. and about 50° C.

In some embodiments, the matrix is a bead coated with lipid membrane material (e.g., Longhi et al., Drug Metabolism and Disposition 39:312-321, 2011), synthetic vesicles, liposomes or micelles. In some embodiments, the synthetic vesicles are formed from mixtures of phosphatidylcholine, ceramide, phosphatidylethanolamine or phosphatidylserine. In other embodiments, the synthetic vesicles are formed from mixtures of sodium dodecylsulfate, cetyltrimethylammonium bromide, sodium octylsulfate, octyl trimethyl ammonium bromide, 1-palmitoyl-2-oleyl-sn-glycero-30-phosphocholine or bis (2-ethylhexyl) sodium sulfosuccinate. In still other embodiments, the synthetic vesicles are formed from mixtures of sodium dodecylsulfate and cetyltrimethylammonium sulfate. In still other embodiments, micelles are formed from sodium dodecylsulfate and cetyltrimethylammonium bromide. In still other embodiments, synthetic vesicles are coated with carbohydrates, charged compounds or proteins. In still other embodiments, the matrix is an octanol/water mixture. In general, the lipid matrix must be reproducibly synthesized, be stable to the experimental conditions and have a dynamic range sufficient to resolve compounds of differing polarity. As such, the selection of a proper lipid matrix can involve routine experimentation, which is well within the ambit of the skilled artisan.

Compounds absorbed by the matrix may be separated from compounds not absorbed by the matrix by methods including, but not limited to, centrifugation, filtration, electrophoresis or application of a magnetic field to magnetic beads. In some embodiments, the amounts of compounds absorbed by the matrix and/or the amounts of compounds not absorbed by the matrix are measured by absorbance, fluorescence, radioactivity or quantitative mass spectrometry. In other embodiments, the amounts of compounds absorbed by the matrix and/or amounts of compounds not absorbed by the matrix are measured by DNA sequencing or qPCR.

In some embodiments, a method of estimating LogD of one compound which includes a ligand operatively linked to a recognition element is provided. The method comprises contacting the compound with a gel matrix, applying a voltage gradient to the gel matrix and measuring the R_{f} of the compound on the gel matrix wherein the R_{f} of the compound on the gel matrix provides an estimate of LogD of the compound.

In some embodiments, a method of estimating LogD of two or more compounds which include a ligand operatively linked to a recognition element is provided. The method includes the steps of contacting the compounds with a gel matrix, applying a voltage gradient to the gel matrix and measuring the R_{f} of the compounds on the gel matrix wherein the R_{f} of the compounds on the gel matrix provides an estimate of LogD of the compounds.

In some embodiments, the gel matrix includes a lipid phase. In other embodiments, the lipid phase is synthetic vesicles, liposomes or micelles. In still other embodiments, compounds on the gel matrix are detected by staining. In still other embodiments, compounds on the gel matrix are detected by spectroscopic means (e.g., fluorescence or absorbance).

In some embodiments, discrete regions of the gel matrix which include compounds are isolated. The compounds are eluted from each discrete isolated region of the gel matrix to provide a unique fraction, which is bar coded with a unique oligonucleotide, the fractions are pooled, amplified by the polymerase chain reaction and sequenced by NextGen sequencing.

In other embodiments, compounds are eluted from each discrete isolated region of the gel matrix to provide a unique fraction. The fractions are amplified by the polymerase chain reaction and sequenced by NextGen sequencing

In some embodiments, a method of estimating LogD of one compound which include a ligand operatively linked to a recognition element is provided. The method includes the steps of contacting the compound with a chromatographic matrix and measuring the retention times of the compound on the chromatographic matrix wherein the measured retention times of the compound on the chromatographic matrix provides an estimate of LogD of the compound.

In some embodiments, a method of estimating LogD of two or more compounds which include a ligand operatively linked to a recognition element is provided. The method includes the steps of contacting the compounds with a chromatographic matrix and measuring the retention times of the compounds on the chromatographic matrix wherein the measured retention times of the compounds on the chromatographic matrix provides an estimate of LogD of the compounds.

Selection of a proper chromatographic matrix is within the ambit of the skilled artisan and is often a matter of trial and error. Elution with discrimination of recognition elements operatively linked to ligands is an essential requirement of a chromatographic matrix. Another feature of importance is pore size. In some embodiments, the pore size of the chromatographic matrix is about 100 Å. In other embodiments, the pore size of the chromatographic matrix is about 4000 Å.

In some embodiments, the recognition element is a single stranded DNA oligonucleotide, the ligand is a peptide or organic molecule and the chromatographic matrix is reverse phase chromatographic support. In other embodiments, the retention time of the compounds is chromₛₛₓₓLogD wherein xx is the number of nucleotides in the DNA oligonucleotide. In some embodiments, xx is 20. In still other embodiments, two or more standard compounds are included in the contacting step where the standard compounds include ligands of measured chromₛₛₓₓLogD and measured chromLogD. In still other embodiments, the chromatographic matrix is packed into a high pressure liquid chromatography column In still other embodiments, fractions including the compounds are collected by elution of the column. In still other embodiments, each fraction is amplified by the polymerase chain reaction and sequenced by NextGen sequencing to provide an estimate of the retention time of the compounds in the fraction. In still other embodiments the retention times of the compounds are compared with the retention times of the standard compounds to provide an estimate of chromₛₛₓₓLogD of the compounds. In still other embodiments, a unique oligonucleotide bar code is attached to compounds in each fraction, the fractions are pooled, amplified by the polymerase chain reaction and sequenced by NextGen sequencing to provide an estimate of the retention time of the compounds in the fractions. In still other embodiments the retention times of the compounds are compared with the retention times of the standard compounds to provide an estimate of chromₛₛₓₓLogD of the compounds.

In some embodiments, the recognition element is double stranded DNA, the ligand is a peptide or organic molecule and the chromatographic matrix is a reversed phase chromatographic support. In some embodiments, the retention time of the compounds is chrom_{dsxx}LogD. In still other embodiments, xx is 220. In still other embodiments, two or more standard compounds are included in the contacting step where the standard compounds include ligands of measured chrom_{dsxx}LogD and measured chromLogD. In still other embodiments, the chromatographic matrix is packed into a high pressure liquid chromatography column. In still other embodiments, fractions including the compounds are collected by elution of the column. In still other embodiments, each fraction is amplified by the polymerase chain reaction and sequenced by NextGen sequencing to provide an estimate of the retention time of the compounds in the fraction. In still other embodiments the retention times of the compounds are compared with the retention times of the standard compounds to provide an estimate of chrom_{dsxx}LogD of the compounds. In still other embodiments, a unique oligonucleotide bar code is attached to compounds in each fraction, the fractions are pooled, amplified by the polymerase chain reaction and sequenced by NextGen sequencing to provide an estimate of the retention time of the compounds in the fractions. In still other embodiments the retention times of the compounds are compared with the retention times of the standard compounds to provide an estimate of chrom_{dsxx}LogD of the compounds.

Those of skill in the art will appreciate that, when the recognition element is capable of binding a complementary moiety (e.g., oligonucleotide, single stranded DNA or RNA) that the lipophilicity or LogD of a compound which includes a ligand operatively linked to a recognition element may be estimated in a number of different configurations, including, but not limited, to the following. First, the lipophilicity or LogD of a compound which includes a ligand operatively linked to a recognition element may be estimated without any modification. Second, the lipophilicity or LogD of a compound which includes a ligand operatively linked to a recognition element may be estimated after binding to a complementary moiety. Third, the lipophilicity or LogD of a compound which includes a ligand operatively linked to a recognition element may be estimated after binding to a modified complementary moiety, such as, for example, a complementary moiety with an attached group, which in some embodiments may be hydrophobic.

In some embodiments, the recognition element is an oligonucleotide, single stranded DNA or single stranded RNA, which is operatively linked to a first ligand at the 3′ terminus. In some of these embodiments, where the oligonucleotide, single stranded DNA or single stranded RNA is operatively linked to the first ligand at the 3′ terminus is hybridized with a complementary oligonucleotide or single stranded DNA operatively linked to a second ligand at the adjacent 5′ terminus prior to contacting the mixture of compounds with the lipid matrix.

In some embodiments, the recognition element is an oligonucleotide, single stranded DNA or single stranded RNA, which is operatively linked to a first ligand at the 5′ terminus. In some of these embodiments, where the oligonucleotide, single stranded DNA or single stranded RNA is operatively linked to the first ligand at the 5′ terminus is hybridized with a complementary oligonucleotide or single stranded DNA operatively linked to a second ligand at the adjacent 3′ terminus prior to contacting the mixture of compounds with the matrix. It should be understood that, in some embodiments, the first ligand can be at the 3′ terminus and the second ligand may be at the 3′ terminus.

When the hybridized compound, supra, is part of a combinatorial library, the second ligand may be, but is not necessarily identical, for all members of the library. In some embodiments, the second ligands of the combinatorial library, supra, have similar octanol-water coefficients (log D). In other embodiments, the second ligands may have octanol-water coefficients (log D) greater than 1, greater than 2, greater than 3, greater than 4, greater than 5 or greater than 6. In some embodiments, the second ligands of the combinatorial library, supra, are identical. In other of these embodiments, the second ligands are hydrophobic. In still other of these embodiments, the second ligands have octanol-water coefficients (log D) greater than 5 or greater than 6. In still other of these embodiments, the second ligands are C₆-C₃₀ alkanyl, C₁₈ alkanyl, aryl, arylalkyl, sterol derivatives, steroid derivatives or cholesterol derivatives.

Without wishing to be bound by theory, a hydrophobic 3′ constant second ligand, above, may confer measurable distribution of double stranded DNA into a membrane fraction which may in the middle of the dynamic range of a membrane based assay. The proximity of the 5′ variable first ligand may perturb the affinity of the 3′ constant second ligand for the membrane fraction thus, in principle, allowing for measurement of the lipophilicity of the 5′ variable first ligand and in some embodiments, LogD of the 5′ variable first ligand.

In some embodiments, a double stranded DNA molecule comprising one oligonucleotide operatively linked to a variable first ligand at the 3′ terminus hybridized to a complementary oligonucleotide operatively linked to a constant second ligand at the adjacent 5′ terminus where the LogD of the second ligand is greater than 6 is provided.

In an eighth aspect, above molecule is used in a method of estimating LogD of two or more first ligands of double stranded DNA molecules. The method includes the steps of contacting the double stranded DNA molecules with a lipid matrix, separating the double stranded DNA molecules absorbed by the lipid matrix from double stranded DNA molecules not absorbed by the lipid matrix and measuring the amounts of double stranded DNA molecules absorbed by the lipid matrix and/or the amounts of double stranded DNA molecules not absorbed by the matrix wherein measurement of the amounts of double stranded DNA molecules absorbed by the matrix and/or the amounts of the double stranded DNA molecules not absorbed by the matrix provide an estimate of LogD of the first ligands of the double stranded DNA molecules.

In any of the above methods, use of at least two standard compounds, which in some embodiments are operatively linked to recognition elements may provide estimates of LogD of unknown compounds, which include a ligand operatively linked to a recognition element, by interpolation between the LogD values of the standards. For example, use of a standard compound with a LogD value of 3 and another standard compound with a LogD value of 4 would allow for identification of compounds of LogD between 3 and 4. In another example, standard compounds of known LogD, when attached to recognition elements known in the art and known R_{f}, may be used to bracket the R_{f}, of compounds on a gel matrix and hence to estimate the LogD of the compounds. In still another example, standard compounds of known LogD, may be used to bracket the retention time of the compounds on the chromatographic matrix and hence to estimate LogD of the compounds.

In any of the above methods, compounds, which include a ligand operatively linked to a recognition element, may be affinity purified by binding to a target, which may be a biological target, such, as for example, a receptor, an enzyme, a protein, a cell, a membrane preparation, etc., prior to contacting with a matrix. In some embodiments, affinity purification enriches the mixture of compounds by removing non-binding members, hence providing lipophilicity estimates only for compounds which have demonstrated affinity for the target.

The skilled artisan will also appreciate that the above methods may be used in either a selection mode or a screening mode. As an example of a selection mode, a library of compounds may be applied to a gel matrix including a lipid phase, separated by electrophoresis, desired regions of the gel isolated and the nucleic tags on the corresponding compounds PCR amplified. Sequencing and subsequent correlation of sequence with mobility on the gel matrix enables assignment of LogD to members of the compound library in the selected region of the gel. Alternatively, a library of compounds may be contacted with a lipid matrix, the population that is bound to the lipid matrix separated, PCR amplified and used as input for another round of translation and selection on a lipid matrix. In still other embodiments, a library of compounds may be contacted with a chromatographic matrix, fractions collected, PCR amplified and used as input for another round of translation and selection on a chromatographic matrix. Iteration of such methods may exponentially enrich for compounds with a desired LogD. In some embodiments, the PCR amplified material may be used as input, for example, affinity selection.

The above methods can also be used to measure the LogD of a single compound which includes a ligand operatively linked to a recognition element as well as compounds that are members of a combinatorial library (i.e., a complex mixture).

In some embodiments, a plurality of compounds, each linked to a unique oligonucleotide are synthesized and pooled. The pooled compounds may now be separated on the basis of differential LogD using any of the methods described above. Sequencing of the collected fractions provides the relative abundance of any given oligonucleotide in any given fraction, which then enables a calculation to estimate LogD. Because each compound is attached to a unique oligonucleotide a plurality of compounds can be assayed in parallel. The pooled compounds may affinity purified, for example, against an immobilized target, prior to separation by the methods described herein.

In some of the above embodiments, a mixture of compounds is prepared by synthesizing compounds attached to an invariant oligonucleotide in separate vessels by methods know in the art, supra. The invariant oligonucleotide may be primed with unique oligonucleotides that include a unique bar code region interposed between flanking oligonucleotide regions identical to the above invariant oligonucleotide and converted to double stranded material by conventional methods. The double stranded material is then pooled and used in any of the above methods to provide estimates of LogD for the compounds. In some embodiments, the double stranded material may be affinity purified against a biological target, before being used in any of the methods described above.

It should be noted that any of the above methods may be used to provide relative lipophilicities of combinatorial library compounds without estimation of LogD when standard compounds are not used or are unavailable. In many of the above methods standard compounds are included in the contacting steps.

In some of the above embodiments, the ligand is an oligonucleotide, single stranded RNA, single stranded DNA, double stranded RNA, double stranded DNA, a peptide, a depsipeptide, a peptoid or an organic compound of molecular weight of less than 2000 daltons. In other embodiments, the ligand is a peptide, a peptoid or an organic compound of molecular weight of less than 2000 daltons. In still other embodiments, the ligand is a peptide or an organic compound of molecular weight of less than 2000 daltons.

The ligand is operatively linked to a recognition element with a linker, which generally is any molecule or substance which performs the function of connecting the ligand to the recognition element. The distance between the ligand and the recognition element may be greater than about 10 Å, about 25 Å, about 50 Å or about 100 Å.

The linker may vary in structure and length. The linker may be hydrophobic or hydrophilic, long or short, rigid, semi rigid or flexible, etc. The linking group can comprise, for example, a polymethylene chain, such as a (CH₂)ₙ — chain or a poly(ethylene glycol) chain, such as a —(CHₙCH₂O)ₙ chain, where in both cases n is an integer from 1 to about 20, 5′-O-Dimethoxytrityl-1′,2′-Dideoxyribose-3′-[(2-cyanoethyl)-(N,N-diisopropyl)]-phosphoramidite; 9-O-Dimethoxytrityl-triethylene glycol,1-1-[(2-cyanoethyl)-(N,N-isopropyl)]-phosphoramidite; 3-(4,4′-Dimethoxytrityloxy)propyl-1-1[(2-cyanoethyl)-(N,N-diisopropyl)]-phosphoramidite; and 18-O-Dimethoxytritylhexaethyleneglycol,1,-[(2-cyanoethyl)-(N,N-diisopropyl)]1-phosphoramidite, amino-carboxylic linkers (e.g., peptides (e.g., Z-Gly-Gly-Gly-Osu or Z-Gly-Gly-Gly-Gly-Gly-Gly-Osu), PEG (e.g., Fmoc-aminoPEG2000-NHS or amino-PEG (12-24)-NHS), or alkane acid chains (e.g., Boc-ε-aminocaproic acid-Osu)), click chemistry linkers (e.g., peptides (e.g., azidohomalanine-Gly-Gly-Gly-OSu or propargylglycine-Gly-Gly-Gly-OSu), PEG (e.g., azido-PEG-NHS), or alkane acid chains (e.g., 5-azidopentanoic acid, (S)-2-(azidomethyl)-1-Boc-pyrrolidine, or 4-azido-butan-1-oic acid N-hydroxysuccinimide ester)), thiol-reactive linkers (e.g., PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]propionamido)hexanoate))), amidites for oligonucleotide synthesis (e.g., amino modifiers (e.g., 6-(trifluoroacetylamino)-hexyl-(2-cyanoethyl)-(N,N-diisopropyl)-phosphoramidite), thiol modifiers (e.g., S-trityl-6-mercaptohexyl-1-[(2-cyanoethyl)-(N,N-diisopropyl)]-phosphoramidite, or chick chemistry modifiers (e.g., 5-hexynl-TTT(T)₀₋₇, 6-hexynl-TTT(T)₀₋₇, 5-hexyn-1-yl-(2-cyanoethyl)-(N,N-diisopropyl)-phosphoramidite, 6-hexyn-1-yl-(2-cyanoethyl)-(N,N-thisopropyl)-phosphoramidite, 3-dimethoxytrityloxy-2-(3-(3-propargyloxypropanamido)propanamido)propyl-1-O-succinoyl, long chain alkylamino CPG or 4-azido-butan-1-oic acid N-hydroxysuccinimide ester)). Additional examples of linkers are provided, infra.

The recognition element, in broadest terms, may be an oligonucleotide, a double stranded oligonucleotide, single stranded RNA, single stranded DNA, double stranded DNA, a double stranded RNA-DNA hybrid, a DNA binding protein, a RNA binding protein, a peptide nucleic acid, a peptide, a depsipeptide, a polypeptide, locked nucleic acids, an antibody or a peptoid. In some embodiments, the recognition element is an oligonucleotide, a double stranded oligonucleotide, single stranded RNA, a double stranded RNA-DNA hybrid, single stranded DNA, double stranded DNA or a peptide nucleic acid. In other embodiments, the recognition element is an oligonucleotide, single stranded DNA, single stranded RNA or double stranded DNA.

In some embodiments, the compounds described above may be members of combinatorial libraries and the method may simultaneously provide estimates of the lipophilicity of more than one member(s) of those particular combinatorial libraries. Combinatorial libraries include, but are not limited to, tagged combinatorial libraries described, supra.

As will be appreciated by the skilled artisan, the recognition element may include, but is not limited to, all tags or labels previously described in the art and all methods used to prepare such tags. In some embodiments, the precise chemical structure of the recognition element will not be of determinative importance in the methods described herein. Accordingly, the methods described herein may be used with any tagged combinatorial library, including those not yet known in the art.

As is known to the skilled artisan, identification of the compound(s) operatively linked to recognition element(s) can be accomplished by identification of the recognition element(s). Generally, recognition elements may be identified by any method know to those of skill in the art including, for example, but not limited to, biological methods (e.g., affinity binding, sequencing, etc.) and chemical methods (e.g., NMR, mass spectroscopy, etc.). In some embodiments, when the recognition element is a nucleic acid, identification involves amplification and sequencing amplified recognition elements or quantitative hybridization to complementary sequences Amplification of nucleic acids, sequencing of nucleic acids and quantitative hybridization are conventional and are well known in the art.

In still another aspect, a compound of Formula (I) is presented:

RE-(X₁)—(C₁X₂)ₙ—C₂X₃)ₒ-Lₐ (I)

or salts, hydrates, or solvates thereof wherein:

RE is a recognition element;

Lₐ is a ligand;

C₁ and C₂ are independently connecting elements;

n and o are independently 0 or 1; and

X₁, X₂ and X₃ are functional groups.

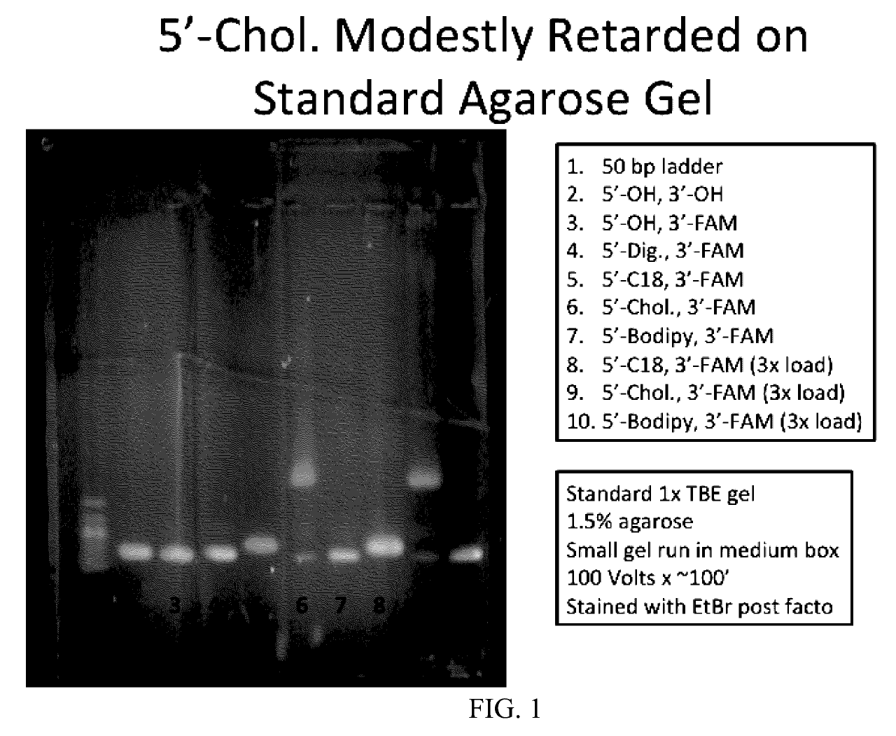

In some embodiments, the compound of Formula (I) is a coding template used to direct synthesis of small-molecule combinatorial libraries (Harbury, et al., U.S. Pat. No. 7,479,472). Coding templates are compounds having a nucleic acid sequence containing at least one, typically two or more different catenated hybridization sequences, optional constant spacer sequences and an attached linking entity or functional group (i.e., chemical reaction moiety) (FIG. 1). Coding templates are not limited in the number of hybridization sequences and/or constant spacer sequences. The hybridization sequences in any given coding template generally differ from the sequences in any other coding template. It should be noted that different coding templates can share a common codon. The hybridization sequences of each coding template identify the particular chemical compounds used in each successive synthesis step for synthesizing a unique ligand attached to the linking entity or functional group. As such, hybridization sequences of each coding template also identify the order of attachment of the particular chemical units to the linking entity or functional group.

In other embodiments, the compound of Formula (I) is a tagging oligonucleotide, such as those used in methods described in Morgan et al., U.S. Pat. No. 7,972,992; Morgan et al., U.S. Pat. No. 7,935,658; Morgan et al., U.S. Patent Application No. 2011/0136697; Morgan et al., U.S. Pat. No. 7,972,994; Morgan et al., U.S. Pat. No. 7,989,395; Morgan et al., U.S. Pat. No. 8,410,028; Morgan et al., U.S. Pat. No. 8,598,089; Morgan et al., U.S. patent application Ser. No. 14/085,271; Wagner et al., U.S. Patent Application No. 2012/0053901; and Keefe et al., U.S. Patent Application No. 2014/0315762.

In still other embodiments, the compound of Formula (I) is an oligonucleotide described in Pedersen et al., U.S. Pat. No. 7,277,713; Pedersen et al., U.S. Pat. No. 7,413,854; Gouliev et al., U.S. Pat. No. 7,704,925; Franch et al., U.S. Pat. No. 7,915,201; Gouliev et al., U.S. Pat. No. 8,722,583; Freskgard et al., U.S. Patent Application No. 2006/0269920; Freskgard et al., U.S. Patent Application No. 2012/0028812; Hansen et al., U.S. Pat. No. 7,928,211; Hansen et al., U.S. Pat. No. 8,202,823; Hansen et al., U.S. Patent Application No. 2013/0005581; Hansen et al., U.S. Patent Application No. 2013/0288929; Neri et al., U.S. Pat. No. 8,642,514; Neri et al., U.S. Pat. No. 8,673,824; and Neri et al., U.S. Patent Application No. 2014/01288290.

In some embodiments, X₁ is attached to a functional group on RE. In other embodiments, X₂ or X₃ are attached to a functional group on Lₐ. In some of the above embodiments, the functional group on RE is OH, NH₂ or NRₓ where X is an alkyl group or a methyl group. RE and Lₐ are as defined, supra.

In some embodiments, X₁ is —C(O)—, —CONR₁—, —C(O)O—, —P(O)NR₂—, —P(O)(OH)NR₃, —P(O)O—, —P(O)(OH)O—, —S(O)₂NR₄—, —OC(S)—, —OC(O)O—, —OC(S)O—,—OC(O)NR₅—, —NR₆C(O)O—, —NR₇C(O)NR₈—, —OP(O)NR₉—, —OP(O)(OH)NR₁₀—, —OP(O)O—, —OP(O)(OH)O—, —NR₁₁—, —NR₁₂P(O)O—, —NR₁₃P(O)(OH)O—, —S—, —O— or a carbon-carbon bond; X₂ is —C(O)—, —CONR₂₁—, —C(O)O—, —P(O)NR₂₂—, —P(O)(OH)NR₂₃, —P(O)O—, —P(O)(OH)O—, —S(O)₂NR₂₄—, —OC(S)—, —OC(O)O—, —OC(S)O—,—OC(O)NR₂₅—, —NR₂₆C(O)O—, —NR₂₇C(O)NR₂₈—, —OP(O)NR₂₉—, —OP(O)(OH)NR₃₀—, —OP(O)O—, —OP(O)(OH)O—, —NR₃₁—, —NR₃₂P(O)O—, —NR₂₃P(O)(OH)O—, —S—, —O— or a carbon-carbon bond; X₃ is —C(O)—, —CONR₄₁—, —C(O)O—, —P(O)NR₄₂—, —P(O)(OH)NR₄₃, —P(O)O—, —P(O)(OH)O—, —S(O)₂NR₄₄—, —OC(S)—, —OC(O)O—, —OC(S)O—,—OC(O)NR₄₅—, —NR₄₆C(O)O—, —NR₄₇C(O)NR₄₈—, —OP(O)NR₄₉—, —OP(O)(OH)NR₅₀—, —OP(O)O—, —OP(O)(OH)O—, —NR₅₁—, —NR₅₂P(O)O—, —NR₅₃P(O)(OH)O—, —S—, —O— or a carbon-carbon bond; and R₁-R₁₃, R₂₁-R₃₃ and R₄₁-R₅₃ are independently, hydrogen, alkyl, substituted alkyl, aryl, substituted aryl, arylalkyl, substituted arylalkyl, heteroalkyl, substituted heteroalkyl, heteroarylalkyl or substituted heteroarylalkyl. In other embodiments, C₁ and C₂ are independently alkyldiyl, substituted alkyldiyl, aryldiyl, substituted aryldiyl, arylalkyldiyl, substituted arylalkydiyl, heteroalkyldiyl, substituted heteroalkyldiyl, heteroarylalkydiyl or substituted heteroarylalkyldiyl.

In some embodiments, RE is an oligonucleotide, single or double-stranded RNA or single or double-stranded DNA, Lₐ is a peptide, peptoid or an organic compound of molecular weight of less than 2000 daltons, C₁ and C₂ are independently alkyldiyl, substituted alkyldiyl, aryldiyl, substituted aryldiyl, arylalkyldiyl, substituted arylalkydiyl, heteroalkyldiyl, substituted heteroalkyldiyl, heteroarylalkydiyl or substituted heteroarylalkyldiyl, X₁ is —C(O)—, —CONR₁—, —C(O)O—, —P(O)(OH)NR₃— or —P(O)(OH)O—; X₂ is —C(O)—, —CONR₂₁—, —C(O)O—, —P(O)(OH)NR₂₃— or —P(O)(OH)O—; and X₃ is —C(O)—, —CONR₄₁—, —C(O)O—, —P(O)(OH)NR₄₃— or —P(O)(OH)O—. In other embodiments, RE is an oligonucleotide, X₁ is —P(O)(OH)O—, C₁ is —(CH₂)₂O(CH₂)₂O(CH₂)₃, X₂ is —NHC(O)—, o is 0 and Lₐ is cholesterol. In still other embodiments, RE is an oligonucleotide, X₁ is —P(O)(OH)O—, C₁ is —(CH₂)₂O(CH₂)₂O(CH₂)₃, X₂ is —NHP(O)(OH)O— and Lₐ is -nC₁₈H₃₇.

In some embodiments, RE is an oligonucleotide, X₁ is —P(O)(OH)O—, C₁ is —((CH₂)₂O)₂(CH₂)₃—, X₂ is —NHC(O)—, C₂ is n-C₃H₆, n-C₇H₁₄ or n-C₁₁H₂₂ and X₃ is —NHC(O)—. In other embodiments, Lₐ is a peptide, peptoid or an organic compound of molecular weight of less than 2000 daltons. In still other embodiments, Lₐ is a sterol derivative or a cholesterol derivative. In still other embodiments, Lₐ is a compound derived from

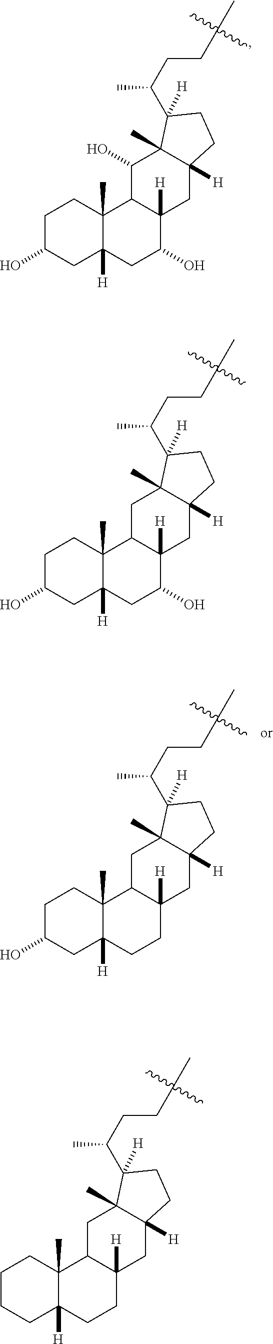

In some embodiments, RE is an oligonucleotide, X₁ is —P(O)(OH)O—, C₁ is —((CH₂)₂O)₂(CH₂)₃—, X₂ is —NHC(O)—, —((CH₂)₂O)₃(CH₂)₂— and X₃ is —NHC(O)—. In other embodiments, Lₐ is a peptide, peptoid or an organic compound of molecular weight of less than 2000 daltons. In still other embodiments, Lₐ is a sterol derivative or a cholesterol derivative. In still other embodiments, Lₐ is a compound derived from

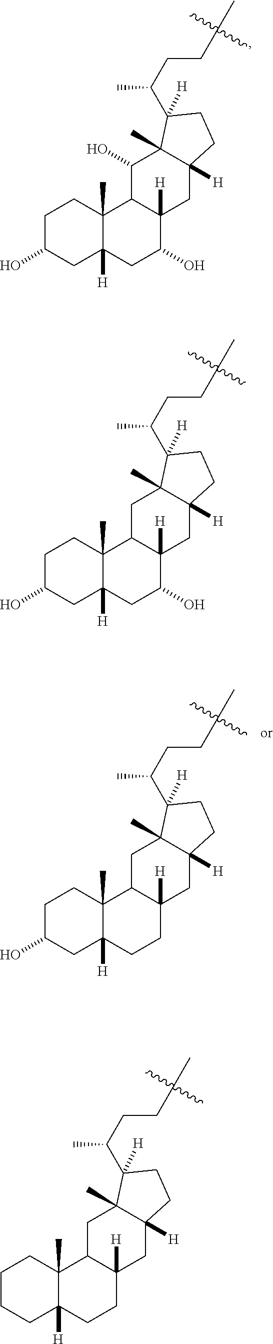

In some embodiments, RE is an oligonucleotide, X₁ is —P(O)(OH)O—, C₁ is —((CH₂)₂O)₂(CH₂)₃—, X₂ is —NHC(O)—, C₂ is piperdinyl or —(CH₂)₃-piperdinyl and X₃ is —C(O)—. In other embodiments, Lₐ is a peptide, peptoid or an organic compound of molecular weight of less than 2000 daltons Lₐ. In still other embodiments, Lₐ is a compound derived from

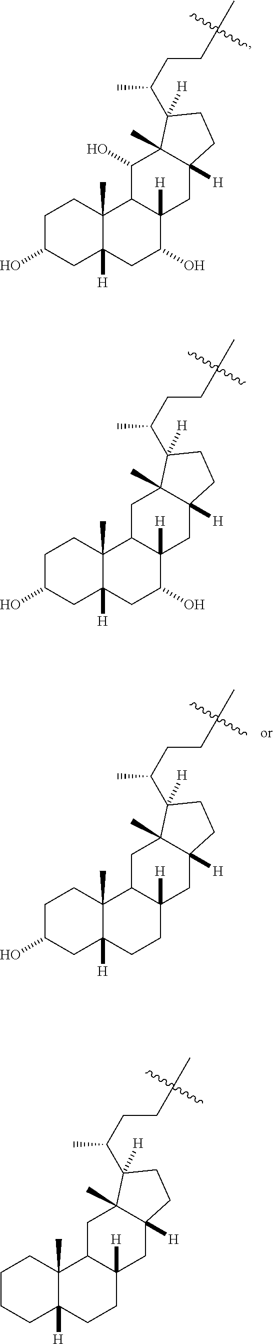

In some embodiments, RE is an oligonucleotide, X₁ is —P(O)(OH)O—, C₁ is —C₆H₁₂—, —C₁₂H₂₄— or C₁₈H₃₆— and X₂ is —NH—. In other embodiments, RE is an oligonucleotide, X₁ is —P(O)(OH)O—, C₁ is —((CH₂)₂O)₅CH₂CH₂—, X₂ is —P(O)(OH)O—, L₂ is C₁₂H₂₄, and X₃ is —NH—. In still other embodiments, Lₐ is a peptide, peptoid or an organic compound of molecular weight of less than 2000 daltons.

In many of the above embodiments, a library of compounds including more than one compound of Formula (I) where RE and Lₐ are different is provided. In some embodiments, C₁, C₂, n, o, X₁, X₂ and X₃ are identical in each compound of the library.

In still another aspect, a compound of Formula (II) is presented:

RE_{c}-(X₄)—(C₃X₅)ₖ—(C₄X₆)₁-L_{b} (II)

or salts, hydrates, or solvates thereof wherein:

RE_{c} is a recognition element;

L_{b} is a ligand with similar or identical hydrophobicity;

C₃ and C₄ are independently linkers;

X₄, X₅ and X₆ are functional groups; and

k and 1 or independently 0 or 1.

In some embodiments, X₄ is attached to a functional group on RE. In other embodiments, X₅ or X₆ are attached to a functional group on L_{b}. In some of the above embodiments, the functional group on RE_{c} is OH, NH₂ or NR_{y} where Y is an alkyl group or a methyl group. RE and L_{b} are as defined, supra.

In some embodiments, L_{b} is a peptide or an organic compound of molecular weight of less than 2000 daltons. In other embodiments, L_{b} is a hydrophobic compound with a log P greater than 4, greater than 5 or greater than 6.

In some embodiments, X₄ is —C(O)—, —CONR₆₁—, —C(O)O—, —P(O)NR₆₂—, —P(O)(OH)NR₆₃, —P(O)O—, —P(O)(OH)O—, —S(O)₂NR₆₄—, —OC(S)—, —OC(O)O—, —OC(S)O—,—OC(O)NR₆₅—, —NR₆₆C(O)O—, —NR₆₇C(O)NR₆₈—, —OP(O)NR₆₉—, —OP(O)(OH)NR₇₀, —OP(O)O—, —OP(O)(OH)O—, —NR₇₁—, —NR₇₂P(O)O—, —NR₇₃P(O)(OH)O—, —S—, —O— or a carbon-carbon bond, X₅ is —C(O)—, —CONR₈₁—, —C(O)O—, —P(O)NR₈₂—, —P(O)(OH)NR_{83,} —P(O)O—, —P(O)(OH)O—, —S(O)₂NR₈₄—, —OC(S)—, —OC(O)O—, —OC(S)O—,—OC(O)NR₈₅—, —NR₈₆C(O)O—, —NR₈₇C(O)NR₈₈—, —OP(O)NR₈₉—, —OP(O)(OH)NR₉₀—, —OP(O)O—, —OP(O)(OH)O—, —NR₉₁—, —NR₉₂P(O)O—, —NR₉₃P(O)(OH)O—, —S—, —O— or a carbon-carbon bond, X₅ is —C(O)—, —CONR₉₁—, —C(O)O—, —P(O)NR₉₂—, —P(O)(OH)NR₉₃, —P(O)O—, —P(O)(OH)O—, —S(O)₂NR₉₄—, —OC(S)—, —OC(O)O—, —OC(S)O—,—OC(O)NR₉₅—, —NR₉₆C(O)O—, —NR₉₇C(O)NR₉₈—, —OP(O)NR₉₉—, —OP(O)(OH)NR₁₀₀—, —OP(O)O—, —OP(O)(OH)O—, —NR₁₀₁—, —NR₁₀₂P(O)O—, —NR₁₀₃P(O)(OH)O—, —S—, —O— or a carbon-carbon bond and R₇₁-R₇₃, R₈₁-R₈₃ and R₉₁-R₁₀₃ are independently, hydrogen, alkyl, substituted alkyl, aryl, substituted aryl, arylalkyl, substituted arylalkyl, heteroalkyl, substituted heteroalkyl, heteroarylalkyl or substituted heteroarylalkyl. In other embodiments, C₁ and C₂ are independently alkyldiyl, substituted alkyldiyl, aryldiyl, substituted aryldiyl, arylalkyldiyl, substituted arylalkydiyl, heteroalkyldiyl, substituted heteroalkyldiyl, heteroarylalkydiyl or substituted heteroarylalkyldiyl.

In some embodiments, RE_{c} is an oligonucleotide, single stranded RNA or single stranded DNA, L_{b} is a peptide or an organic compound of molecular weight of less than 2000 daltons, C₁ and C₂ are independently alkyldiyl, substituted alkyldiyl, aryldiyl, substituted aryldiyl, arylalkyldiyl, substituted arylalkydiyl, heteroalkyldiyl, substituted heteroalkyldiyl, heteroarylalkydiyl or substituted heteroarylalkyldiyl, X₄ is —C(O)—, —CONR₁—, —C(O)O—, —P(O)(OH)NR₃— or —P(O)(OH)O—; X₅ is —C(O)—, —CONR₂₁—, —C(O)O—, —P(O)(OH)NR₂₃— or —P(O)(OH)O— and X₆ is —C(O)—, —CONR₄₁—, —C(O)O—, —P(O)(OH)NR₄₃— or —P(O)(OH)O—. In some embodiments, L_{b} is C₆-C₃₀ alkanyl, C₁₈ alkanyl, aryl, arylalkyl, a sterol derivative, a steroid derivative or a cholesterol derivative.

In many of the above embodiments, a library of compounds including more than one compound of Formula (II) where RE_{c} is different and L_{b} is of similar hydrophobicity is provided. In some embodiments, C₃, C₄, k, l, X₄, X₅ and X₆ are identical in each compound of the library.

In still another aspect a compound comprising a compound of Formula (I) hybridized to a compound of Formula (II) wherein RE and RE_{c} are independently an oligonucleotide, single stranded RNA or single stranded DNA, C₁, C_{2,} n, o, X₁, X₂, X₃, Lₐ, L_{b} C₃, C₄, k, l, X₄, X₅ and X₆ are as defined above is provided. In some embodiments, a library of compounds including more than one compound of Formula (I) hybridized to a compound of Formula (II) is provided. In other embodiments, compounds where RE, RE_{c} and Lₐ are different and L_{b} is of similar hydrophobicity is provided. In other embodiments, L_{b} is of similar hydrophobicity, C₁, C₂, n, o, X₁, X₂, X₃, C₃, C₄, k, l, X₄, X₅ and X₆ are identical in each compound of the library. In still other embodiments, L_{b}, C₁, C₂, n, o, X₁, X₂, X₃, C₃, C₄, k, l, X₄, X₅ and X₆ are identical in each compound of the library.

Specific compounds are illustrated in Table 1 below. The recognition elements are DNA oligonucleotides with connectivity to the ligand or linkers to the 5′end of the oligomers.

TABLE I

| No. | RE | Structure of ligand and/or linkers(s) |
|---|---|---|
| 1 | X | 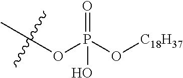 |
| 2 | X | 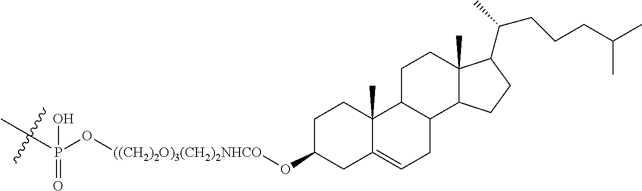 |
| 3 | Y | 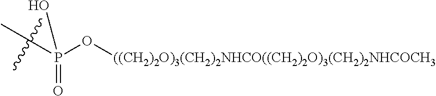 |
| 4 | Y | 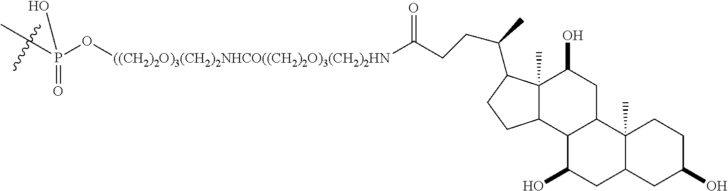 |
| 5 | Y | 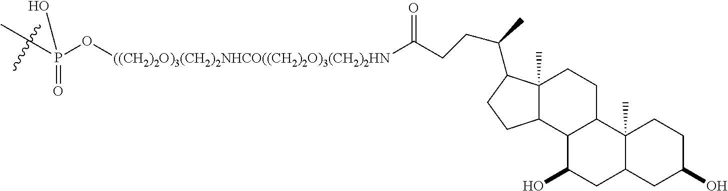 |
| 6 | Y | 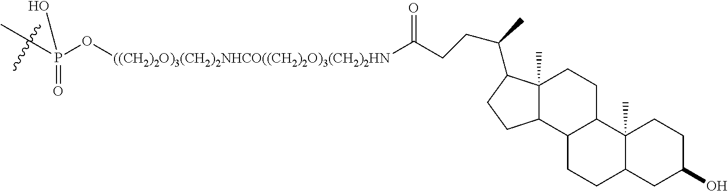 |
| 7 | Y | 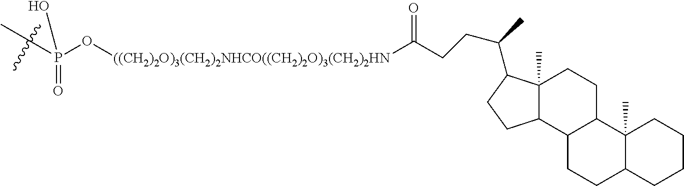 |
| 8 | Y | 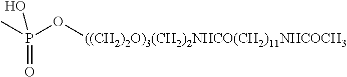 |
| 9 | Y | 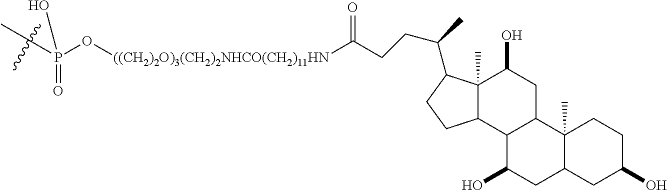 |
| 10 | Y | 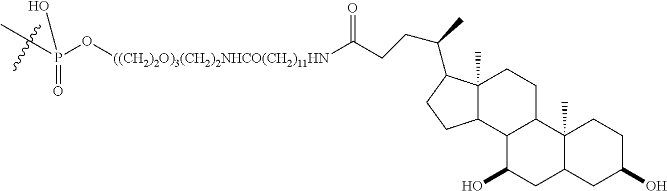 |
| 11 | Y | 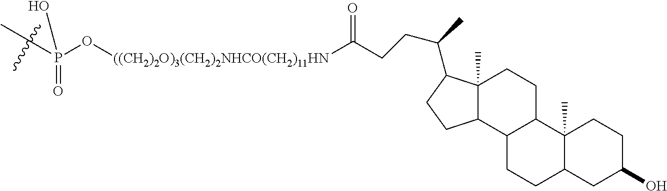 |
| 12 | Y | 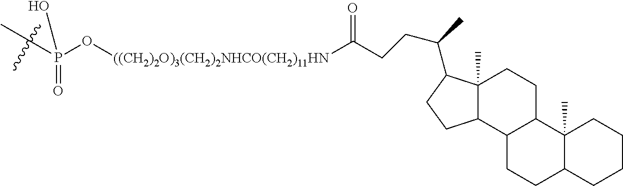 |
| 13 | Y | 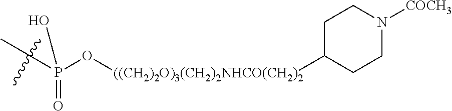 |
| 14 | Y | 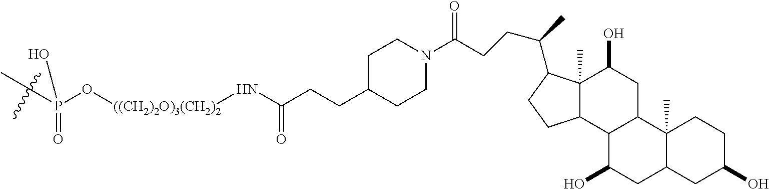 |
| 15 | Y | 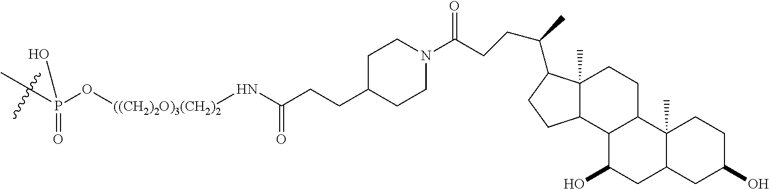 |
| 16 | Y | 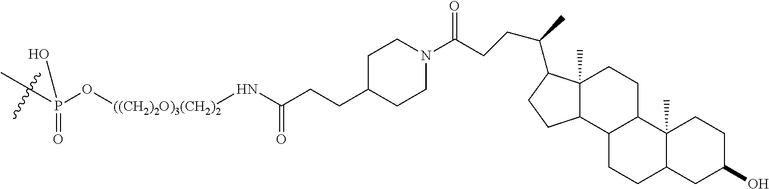 |
| 17 | Y | 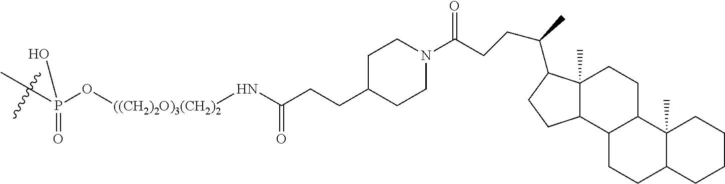 |
| 18 | W | 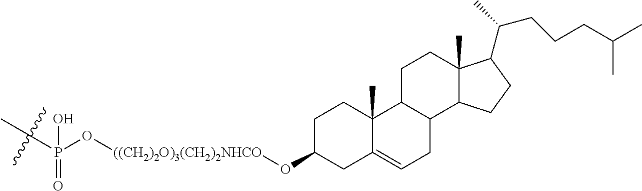 |
| 19 | Z | 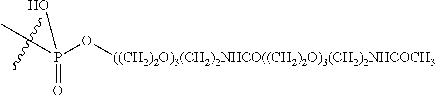 |
| 21 | Z | 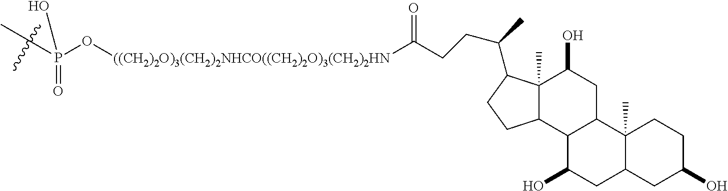 |
| 22 | Z | 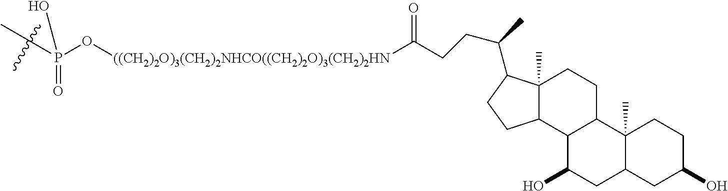 |
| 23 | Z | 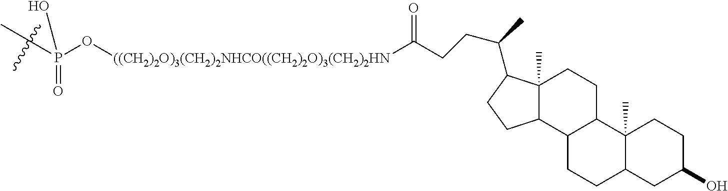 |
| 24 | Z | 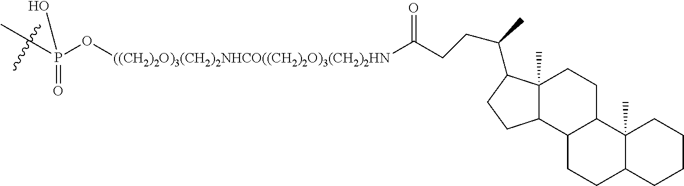 |

X is 5′ GCTCGTCGCATTCGGCACGC-3′ (SEQ. ID. No. 1), Y is ATGGTATXAAGCTTGCCAXAAPPT (SEQ. ID. No. 2), X = pyrrolo dC, W is
GCTCGTCGCATTCGGCACGCCGACAATCATCACACTAGGTCGTGCGTGCGAGACCGCTGTTAATACGGGATAAGGCAGTCCGCTGGTCTCGGATA
GCGCGCTTCCGACACCTTGTATAGGACGCAGCGTGAGACCGACGCACTGGACGACTAGGCTATATTCAGTCGCGACGTGTCCGCGGACGGCTAG
ATGCCTATAATTGTGCCTCCTCACGCGCCGT (SEQ. ID. No. 3), Z is ATGGTATCAAGCTTGCCACAGCCGAAGCAGACTTAATCACGTCGAGC
TCTCTACTGCATAGATTAGCGTACATAGGCCCGGAACCCGGGACAAGGTGTCATCATAGATGTCAGCACTGGGTAGTGGCCTGCAGCTATGTAAA
TCACGCTTGGTAAGTTGGGTAATTCTGTACAGGTCGCGATAATCAGCGGGAATCAGGCGGCAGAATCTCGAGTACTAG (SEQ ID. No. 4).

The following examples are provided for illustrative purposes only and are not intended to limit the scope of the invention.

### EXAMPLES

### Example 1

### Preparation of Fmoc-12-Ado Modified Oligomer

40 uL of a 50% aqueous suspension of DEAE-Sepharose was added to each of 5 wells in a 384-well filter plate. The resin was washed three times with 60 uL 10 mM acetic acid and then spun dry in a centrifuge. 100 nmole ZappT (crude) dissolved in 300 uL 10 mM acetic acid was loaded onto DEAE-Sepharose (5 wells×20 nmole oligo per well), allowed to stand for 10 minutes and then filtered. All further washes were 100 uL, unless stated otherwise.

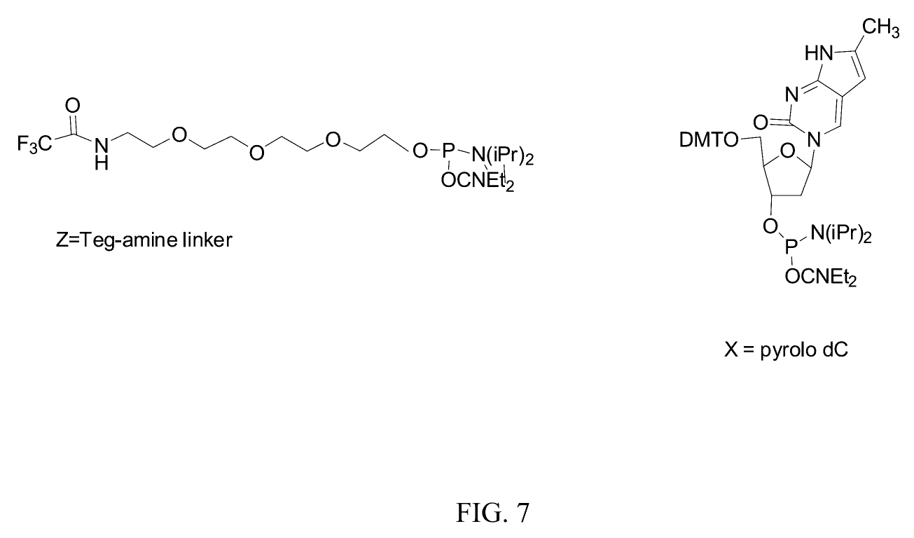

The sequence of “ZappT” is 5′-ZATGGTATXAAGCTTGCCAXA-3′ (SEQ ID. NO. 5) where Z=“TEGamine” and X=pyrrolo dC. “TEGamine”=is a custom 5′ modification that installs a primary amine followed by three polyethylene glycol moieties linked to the 5′ end of the oligonucleotide. “Pyrrolo dC” is a fluorescent deoxycytidine analog that is an ideal probe of DNA structure and dynamics (see http://www.glenresearch.com/Catalog/structural.php#p64). See FIG. 7 for the structural details of “TEGamine” and “pyrrolo dC”.

The resin was washed with water, 100 mM N-methyl morpholine in water, water, 50% water/MeOH, MeOH, 70% MeOH/DMF, 50% MeOH/DMF and spun dry in a centrifuge. In the meantime, a suspension of 12.1 mg Fmoc-12-aminododecanoic acid (Fmoc-12-Ado-OH) in 139 uL DMF (not fully soluble) was prepared. 278 uL N-methyl morpholine solution (10 uL in 909 uL methanol) was added but the solution was still not soluble. The mixture was sonicated but still did not dissolve. The suspension (187.5 uL) was aliquoted and treated with 62.5 uL freshly prepared DMTMM (4-(4,6-dimethoxy-1,3,5-triazin-2-yl)-4-methylmorpholinium) solution (5.2 mg in 157 ul DMF). Then 40 uL aliquots of the resulting suspension were added to each well, which were then kept at room temperature for 1 hour.

The resin was filtered, rinsed with 50% MeOH/DMF, 70% MeOH/DMF, MeOH, 50% MeOH/water, water (allowing to stand 5 minutes), 50% MeOH/water, MeOH, 70% MeOH/DMF, 50% MeOH/DMF and spun down.

Another 187.5 uL Fmoc-12-Ado-OH suspension was treated with 62.5 uL freshly prepared DMTMM solution (6.3 mg in 191 uL DMF). Then 40 uL aliquots of the resulting suspension were added to each well, which were then kept at room temperature for 1 hour. The resin was filtered, rinsed with 50% MeOH/DMF, 70% MEOH/DMF, MeOH, 50% MeOH/water and water.

The oligomer was eluted from each well with 3×33 uL 1.5M NaCl/10 mM ammonium acetate in water, allowed to stand for 2 minutes after each solvent addition prior to filtration. The crude oligomer was purified by RP-HPLC on a Phenomenex Luna C-18 column (Solvent A: 10 mM ammonium acetate in water; Solvent B: acetonitrile). The desired fractions were dried via Speedvac.

### Example 2

### Synthesis of Fmoc-12-amino-4.7.10-Trioxadodecanoic Acid Modified Oligomer

40 uL of a 50% aqueous suspension of DEAE-Sepharose was added to each of 5 wells in a 384-well filter plate. The resin was washed three times with 60 uL 10 mM acetic acid, then spun dry in a centrifuge. 100 nmole ZappT (crude) dissolved in 300 uL 10 mM acetic acid was loaded onto DEAE-Sepharose (5 wells×20nmole oligo per well), allowed to stand for 10 minutes, then filtered. All further washes were 100 uL unless stated otherwise.

The resin was washed with water, 100 mM N-methyl morpholine in water, water, 50% water/MeOH, MeOH, 70% MeOH/DMF, 50% MeOH/DMF and then spun dry in a centrifuge. In the meantime, a solution of 11.2 mg Fmoc-12-amino-4,7,10-trioxadodecanoic acid in 127 uL DMF was prepared. 62.5 uL of this solution was aliquoted and treated with 125 uL N-methyl morpholine solution (10 uL N-methyl morpholine in 909 uL methanol) followed by 62.5 uL freshly prepared DMTMM solution (5.2 mg in 157 ul DMF). Aliquots (40 uL) of the resulting solution were added to each well, which were then kept at room temperature for 1 hour.

The resin was filtered, rinsed with 50% MeOH/DMF, 70% MeOH/DMF, MeOH, 50% MeOH/water, water (allowed to stand for 5 minutes), 50% MeOH/water, MeOH, 70% MeOH/DMF, 50% MeOH/DMF and spun down.

Another 62.5 uL Fmoc-12-amino-4,7,10-trioxadodecanoic acid solution was treated with 125 uL N-methyl morpholine solution then 62.5 uL of freshly prepared DMTMM solution (6.3 mg in 191 uL DMF). Then 40 uL aliquots of the resulting solution were added to each well, which were then kept at room temperature for 1 hour. The resin was filtered, rinsed with 50% MeOH/DMF, 70% MEOH/DMF, MeOH, 50% MeOH/water and water.

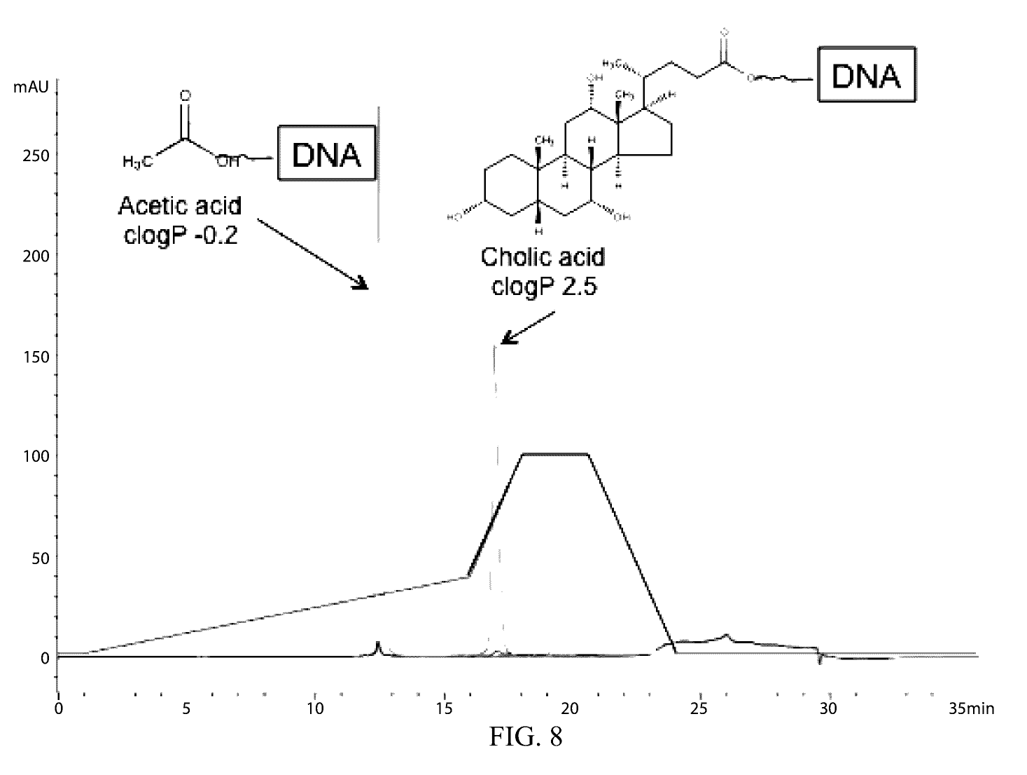

The oligomer was eluted from each well with 3×33 uL of 1.5M NaCl/10 mM ammonium acetate in water, allowed to stand for 2 minutes after each solvent addition prior to filtration. The crude oligomer was purified by RP-HPLC on a Phenomenex Luna C-18 column (Solvent A: 10 mM ammonium acetate in water; Solvent B: acetonitrile). The desired fractions were dried via Speedvac. The resultant compound was analyzed by HPLC essentially as described in FIG. 8 and the purity was judged to be >90% as quantified by the area percent corresponding to the major peak. The measured mass of the compound in the major peak was within experimental error of that predicted for Fmoc-12-amino-4,7,10-trioxadodecanoic acid modified oligomers.

### Example 3

### Synthesis of 4-(1-Fmoc-piperidin-4-yl)butanoic Acid Modified Oligomer

40 uL of a 50% aqueous suspension of DEAE-Sepharose was added to each of 5 wells in a 384-well filter plate. The resin was washed three times with 60 uL 10 mM acetic acid, then spun dry in a centrifuge. 100 nmole ZappT (crude) dissolved in 300 uL 10 mM acetic acid was loaded onto DEAE-Sepharose (5 wells×20 nmole oligo per well), allowed to stand for 10 minutes, then filtered. All further washes were 100 uL, unless stated otherwise.

The resin was washed with water, 100 mM N-methyl morpholine in water, water, 50% water/MeOH, MeOH, 70% MeOH/DMF, 50% MeOH/DMF and then spun dry in a centrifuge. In the meantime, a solution of 12.6 mg 4-(1-Fmoc-piperidin-4-yl)butanoic acid in 159 uL DMF was prepared. 62.5 uL of this solution was aliquoted and treated with 125 uL N-methyl morpholine solution (10 uL N-methyl morpholine in 909 uL methanol) and then 62.5 uL freshly prepared DMTMM solution (4.5 mg in 136 ul DMF). Then, 40 uL aliquots of the resulting solution were added to each well, which were then kept at room temperature for 1 hour.

The resin was filtered, rinsed with 50% MeOH/DMF, 70% MeOH/DMF, MeOH, 50% MeOH/water, water (allowed to stand for 5 minutes), 50% MeOH/water, MeOH, 70% MeOH/DMF, 50% MeOH/DMF and spun down.

Another 62.5 uL 4-(1-Fmoc-piperidin-4-yl)butanoic acid solution was treated with 125 uL N-methyl morpholine solution then 62.5 uL of freshly prepared DMTMM solution (6.3 mg in 191 uL DMF). Then 40 uL aliquots of the resulting solution were added to each well, which were then kept at room temperature for 1 hour. The resin was filtered, rinsed with 50% MeOH/DMF, 70% MEOH/DMF, MeOH, 50% MeOH/water and water.

The oligomer was eluted from each well with 3×33 uL of 1.5 M NaCl/10 mM ammonium acetate in water, allowed to stand for 2 minutes after each solvent addition prior to filtration. The crude oligomer was purified by RP-HPLC on a Phenomenex Luna C-18 column (Solvent A: 10 mM ammonium acetate in water; Solvent B: acetonitrile). The desired fractions were dried via Speedvac.

### Example 4

### General Procedure for Coupling Acetic or Bile Acids to Extended Oligomers

40 uL of a 50% aqueous suspension of DEAE-Sepharose was added to a well in a 384-well filter plate. The resin was washed three times with 60 uL 10 mM acetic acid, then spun dry in a centrifuge. Purified Fmoc-amino acid modified oligomers prepared as described in Examples 1, 2 or 3 was dissolved in 300 uL 10 mM aqueous acetic acid and 60 uL was loaded onto DEAE-Sepharose per well. The suspension was allowed to stand for 10 minutes and filtered. All further washes were 100 uL, unless stated otherwise.

The resin was washed with water, 50% MeOH/water, MeOH, 70% MeOH/DMF, 50% MeOH/DMF, 30% MeOH/DMF and DMF and spun dry in a centrifuge. 60 uL 20% piperidine in DMF was added to the well, and allowed to stand at room temperature for 10 minutes.

The resin was filtered, then washed with DMF, 30% MEOH/DMF, 50% MeOH/DMF, 70% MeOH/DMF, MeOH, 50% MeOH/water, water, 50% MeOH/water, MeOH, 70% MeOH/DMF, 50% MeOH/DMF and then spun dry in a centrifuge.

In the meantime, a 200 mM solution of the carboxylic acid to be attached to the oligomers in DMF was prepared. 12.5 uL of this solution was aliquoted, treated with 25 uL N-methyl morpholine solution (10 uL N-methyl morpholine in 909 uL methanol) and then 12.5 uL freshly prepared DMTMM solution (100 mM in DMF). A 40 uL aliquot of the resulting solution was added to the well, which was kept at room temperature for 1 hour.

The resin was filtered, rinsed with 50% MeOH/DMF, 70% MeOH/DMF, MeOH, 50% MeOH/water, water (allowed to stand 5 minutes), 50% MeOH/water, MeOH, 70% MeOH/DMF, 50% MeOH/DMF and spun down.

Another 12.5 uL of the carboxylic acid solution was treated with 25 uL N-methyl morpholine solution to which was added 12.5 uL freshly prepared DMTMM solution (100 mM in DMF). 40 uL of the resulting mixture was added to the well, which was kept at room temperature for 1 hour, filtered and the resin rinsed with 50% MeOH/DMF, 70% MEOH/DMF, MeOH, 50% MeOH/water and water.

The oligomers were eluted from the well with 3×33 uL 1.5 M NaCl/10 mM ammonium acetate in water and allowed to stand for 2 minutes after each solvent addition prior to filtration. The resulting oligomers were analyzed by HPLC and Nanodrop (for concentration).

Analytical data from an exemplary conjugate is given in FIG. 8. A conjugate of cholic acid with ZappT was prepared and purified essentially as described in Example 4. The trace shows the elution profile of a cholic acid conjugate. Time in minutes is given on the x-axis. Percent Solvent B is given on the y-axis (right side; axis runs from zero to 100 percent). Solvent A was 10 mM ammonium acetate in water. Solvent B was acetonitrile.

### Example 5

### Preparation of Compound 1

Compound 1 with the following sequence Xa-Stearyl: (5′ZGCTCGTCGCATTCGGCACGC-3′) (SEQ. ID NO. 5) was synthesized using by standard methods using phosphoramidites on an automated DNA synthesizer. The Z group was added to oligomers by using a commercially available stearyl phosphoramidite from Glen Research (Catalog No. 10-179-02). This was characterized essentially as described in FIG. 8 and were judged to be >90% pure.

### Example 6

### Preparation of Compound 2

Compound 2 with the following sequence Xa-Cholestryl: (5′Z′ GCTCGTCGCATTCGGCACGC-3′) (SEQ. ID No. 6) was synthesized using by standard methods using phosphoramidites on an automated DNA synthesizer. The Z′ group was added to oligomers by using a commercially available cholestryl phosphoramidite from Glen Research (Catalog No. 10-1976-02). This was characterized essentially as described in FIG. 8 and were judged to be >90% pure.

### Example 7

### Gel Method for Estimation of Lipophilicity of Compounds Operatively Linked to a Recognition Element

Low melt agarose gels (standard 1× TBE, 1.5% agarose), without vesicles were prepared by conventional methods (Molecular Cloning: a Laboratory Manual, T. Maniatis, E. F. Fritsch & J. Sambrook (1982)).

Vesicles are prepared essentially as described by Pascoe et al., Electrophoresis 2003, 24: 4227-4240. Briefly, 0.45 gm of CTAB/SOS (CTAB=hexadecyltrimethylammonium bromide, Sigma catalogue #H9151; SOS=sodium octyl sulfate. Sigma catalogue #O4003)) at a ratio of 3:7 is dissolved in 1× TBE [Trisma-borate-EDTA electrophoresis buffer, Maniatis et al., p. 454] and heated to 50° C. in a water bath to assure dissolution. The solution is cooled overnight to room temperature and then filtered through a 0.2 micron filter. Vesicles prepared in this manner are reported to be stable for multiple days at room temperature.

Low melting agarose (0.75 gm) is dissolved in 25 mL 1× TBE and placed in a water bath at 50° C. along with a separate tube of vesicles. After equilibrium is reached, the tubes are mixed in a preheated flask, gently swirled and poured into a standard horizontal mini-gel apparatus to provide a vesicle agarose gel ((standard 1× TBE, 1.5% agarose, 0.9%).

After the gels have solidified, samples are loaded, run at 100 volts, stained with ethidium bromide and photographed by standard methods (Molecular Cloning; Maniatis et al.; Cold Spring Harbor press, 1982).

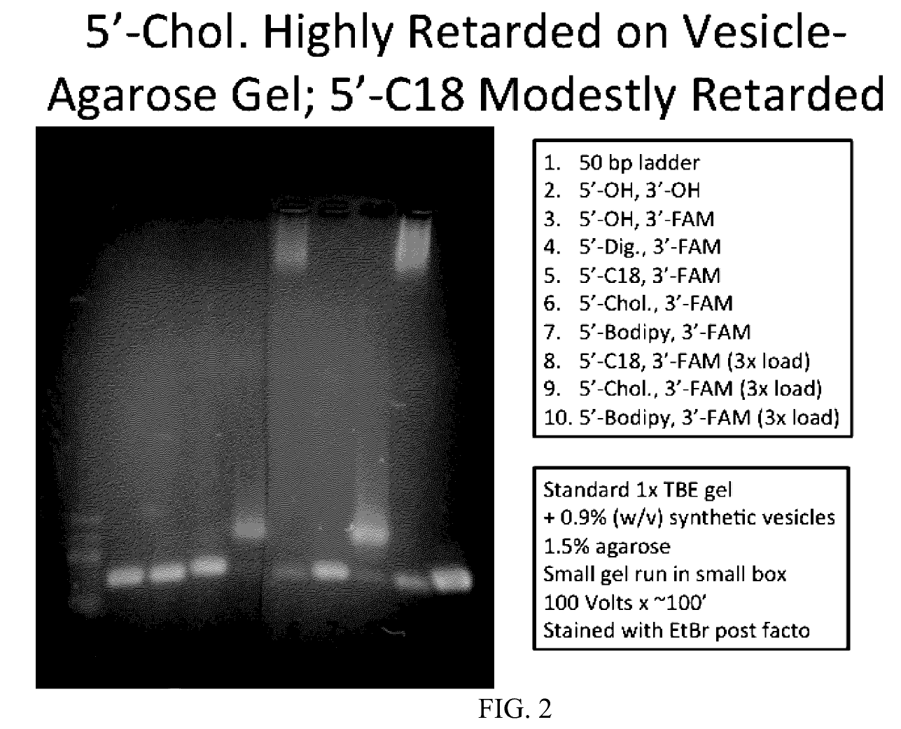

Referring now to FIGS. 1 and 2, the gels indicate that appendage of either cholesterol (Chol) or C₁₈ (n-C₁₈H₃₇) to the 5′ end of a DNA molecule (i.e., compounds 1 and 2, respectively) can greatly retard the mobility of the conjugates in agarose gels containing 0.9% CTAB/SOS vesicles, relative to reference gels lacking these vesicles. FAM is 5′-6-FAM (Fluorescein). The 6-FAM moiety is comprised of a single isomer derivative of fluorescein and is the most commonly used fluorescent dye attachment for oligonucleotides and is compatible with most fluorescence detection equipment. It becomes protonated and has decreased fluorescence below pH 7; it is typically used in the pH range 7.5-8.5. FAM can be attached to 5′ or 3′ end of oligos (https://www.idtdna.com/site/Catalog/Modifications/Product/1108).

Without wishing to be bound by theory, to the degree that mobility is governed by the small molecule and is independent of the DNA sequence of the appended DNA molecule, a surrogate of log K, the partitioning coefficient into the embedded vesicles may be obtained, which may act as a surrogate for diffusion into other lipid membranes. Accordingly, one may in principle, use this method to separate a plurality of DNA molecules with different ligands on gel systems of this type. Sequencing of isolated gel sections can then enable the inference of the mobility of DNA-conjugate species included therein.

### Example 8

### Matrix Method for Estimation of Lipophilicity of Steroids Operatively Linked to a Recognition Element

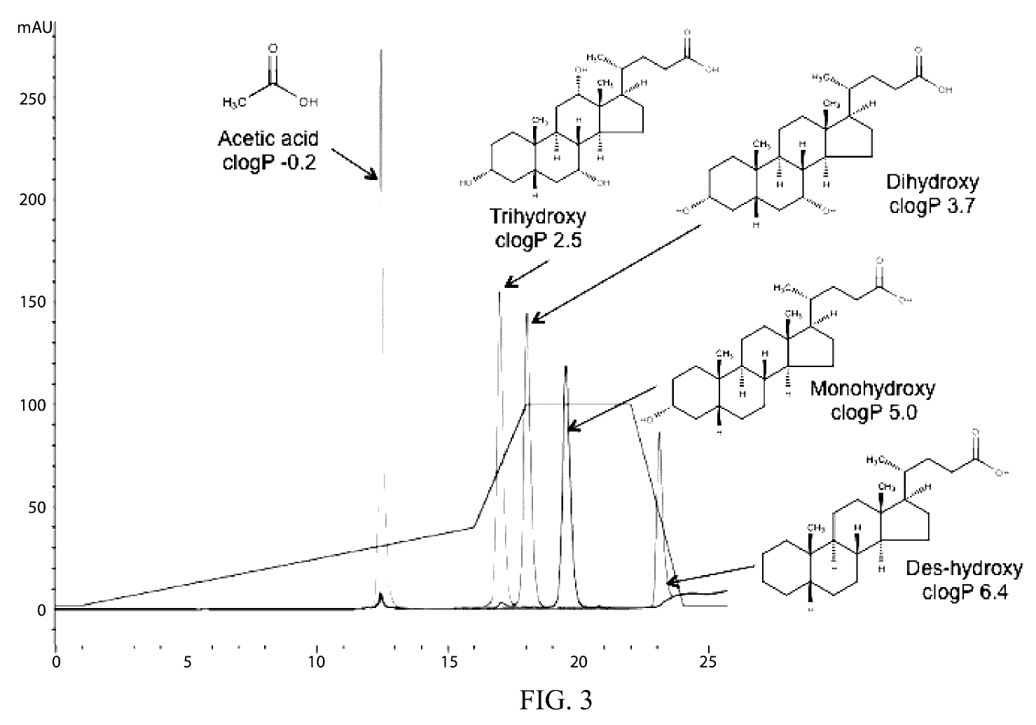

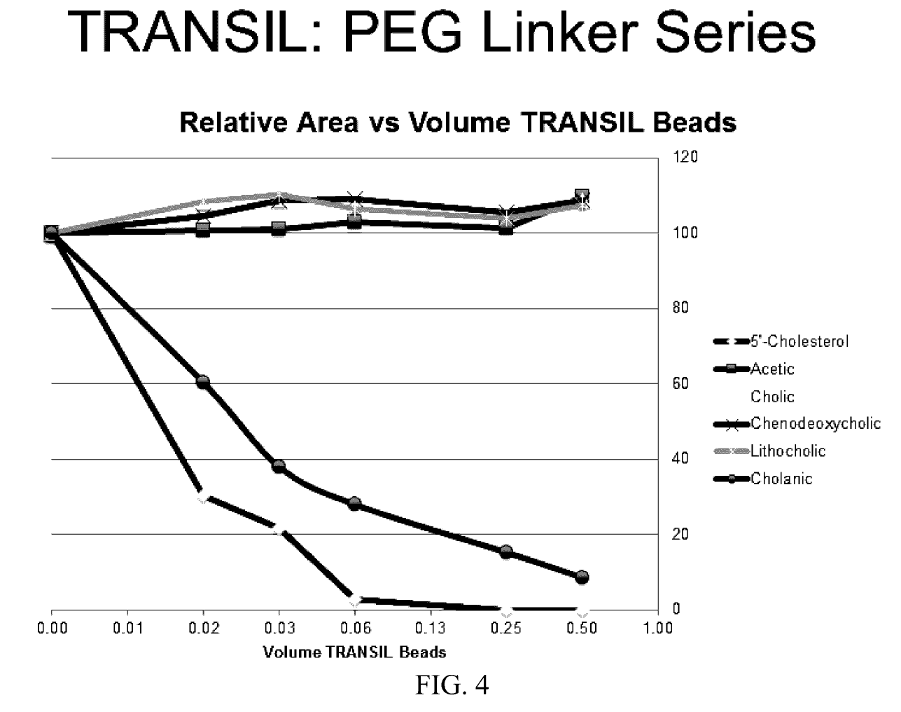

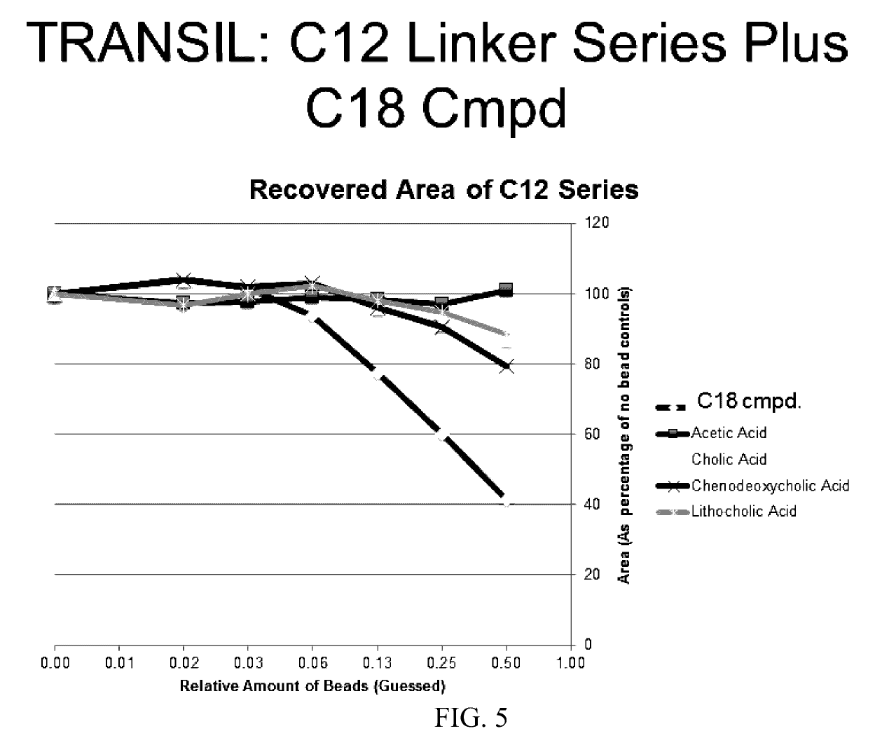

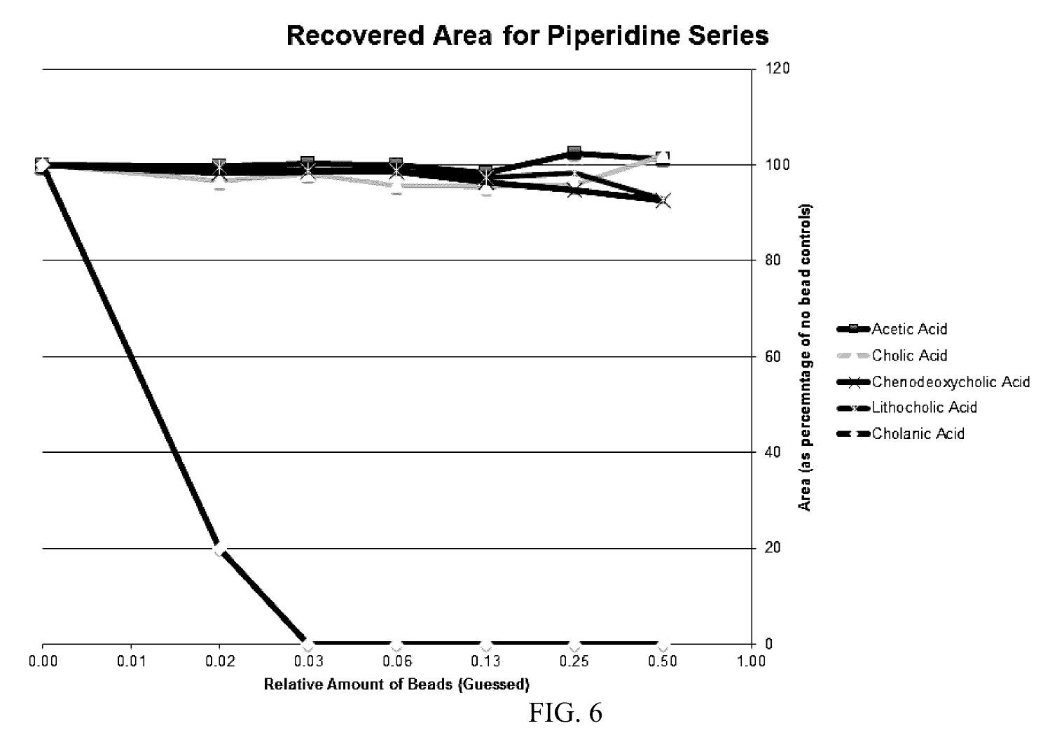

Conjugates ((FIG. 3, Compound 3 is Acetate, Compound 2 is cholesterol, Compound 4 is Cholic, Compound 5 is Chenodeoxycholic, Compound 6 is Lithocholic and Compound 7 is Cholanic), (FIG. 4, Compound 8 is acetate, Compound 9 is Cholic, Compound 10 is Chenodeoxycholic and Compound 11 is Lithocholic), (FIG. 5, Compound 13 is Acetate, Compound 14 is Cholic, Compound 15 is Chenodeoxycholic, Compound 16 is Lithocholic and Compound 17 is Cholanic)), were dissolved in PBS at appropriate concentrations. TRANSIL beads were obtained from ADME Cell, Inc. Alameda, Calif. The vials of TRANSIL beads provided by the kit were thawed to 25° C. and equilibrated in PBS as per the provided protocol. The conjugates were added to TRANSIL beads such that the final concentration of oligo conjugates was ˜200 picomoles/300 microliter. The beads were agitated on a plate shaker at 1000 rpm for 12′ and then spun at 750 g for 10 minutes to pellet the beads and 110 microliters of supernatant was removed, taking care to not take up any TRANSIL beads. The supernatant was analyzed by HPLC-MS according the analytical method of FIG. 3. The oligo-conjugates of interest were quantified by integration of peaks at the known retention times of the conjugates. Two reference tubes with no TRANSIL beads provided the “no bead controls”. The area percentage of each conjugate in each TRANSIL tube that remained in solution was calculated for each sample, using the no bead tubes as the reference. This data was used to generate FIGS. 4, 5 and 6. As indicated by the data, a measurable fraction several of the conjugates distributes into the TRANSIL beads and for those that distribute into beads, the fraction distributing into the beads increases with the bead concentration, as expected.

### Example 9

### Chromatographic Method for Estimation of Lipophilicity of Steroids Operatively Linked to a 20 Base Pair Oligonucleotide

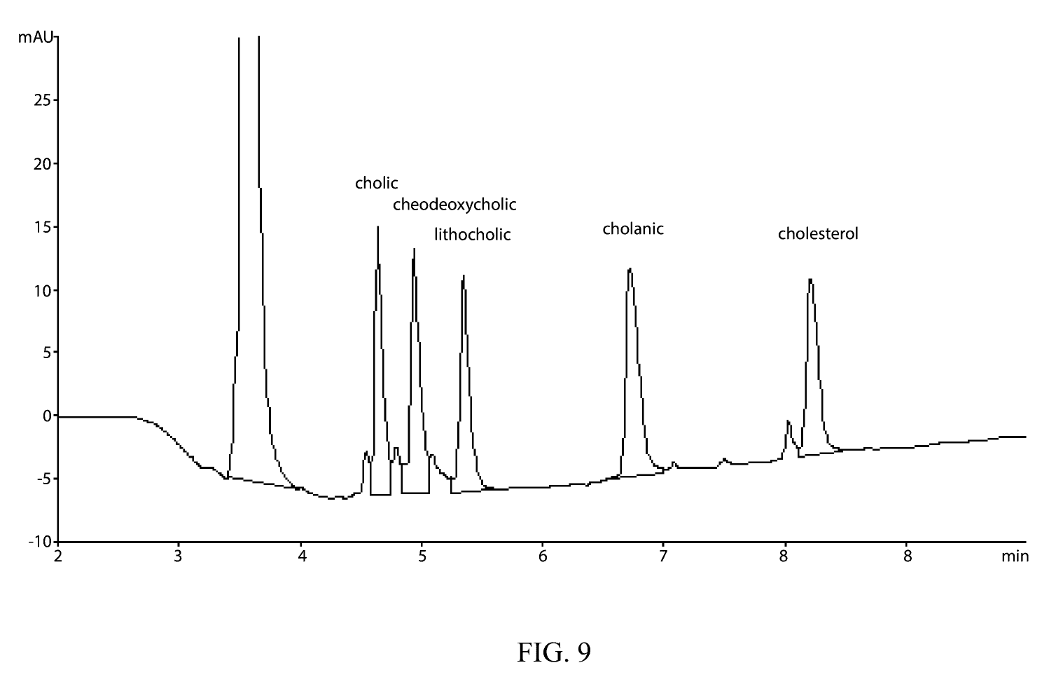

Compounds 3-7 were run on a reverse phase HPLC column, using a Luna C₁₈ column and acetonitrile/ammonium acetate gradient. Solvent A was 10 mM ammonium acetate and solvent B was acetonitrile. Referring now to FIG. 9, the x axis is in minutes and the y axis is % solvent B. As illustrated in FIG. 3, these tool compounds were resolved by HPLC. Accordingly, the rank order of log D can be inferred from the rank order of the retention times on the column Without wishing to be bound by theory, inclusion of appropriate standard compounds may provide useful surrogates of absolute values of log D. It should be also noted that chromLogD and Chrom₂₀LogD may be determined by extensions of the above experiment.

### Example 10

### General Procedure for Conversion of Oligonucleotides of 20 Nucleic Acid Subunits Operatively Linked to a Ligand to Oligonucleotides of 220 Nucleic Acid Subunits Operatively Linked to a Ligand

HPLC purified 20 base pair single stranded oligomers operatively linked to alkyl or steroid ligands were transformed into a double stranded 220 base pair gene via polymerase chain reaction (PCR). The oligomers operatively linked to alkyl or steroid ligands and a 20 base pair reverse oligomer (1 uM) were used as PCR primers in a 100 uL reaction with 10 ng of double stranded 220 base pair gene template with 2× Fusion Flash High-Fidelity PCR Master Mix (ThermoFisher F548S). Thermocycling was performed at 98 C.° for 10 s, followed by 10 cycles of 98 C°, 1 s, 55 C°, 5 s, 72 C°, 10 s then followed by an additional protocol of 98 C.° for 10 s followed by 15 cycles of 98 C°, 1 s, 50 C°, 5 s, 72 C°, 10 s. PCR products of 220 base pair were confirmed via gel electrophoresis, purified with the QIAquick PCR Purification Kit (Qiagen) and DNA concentration was quantified via nanodrop A260 measurements.

### Example 11

### Chromatographic Method for Estimation of Lipophilicity of Steroids Operatively Linked to a Recognition Element

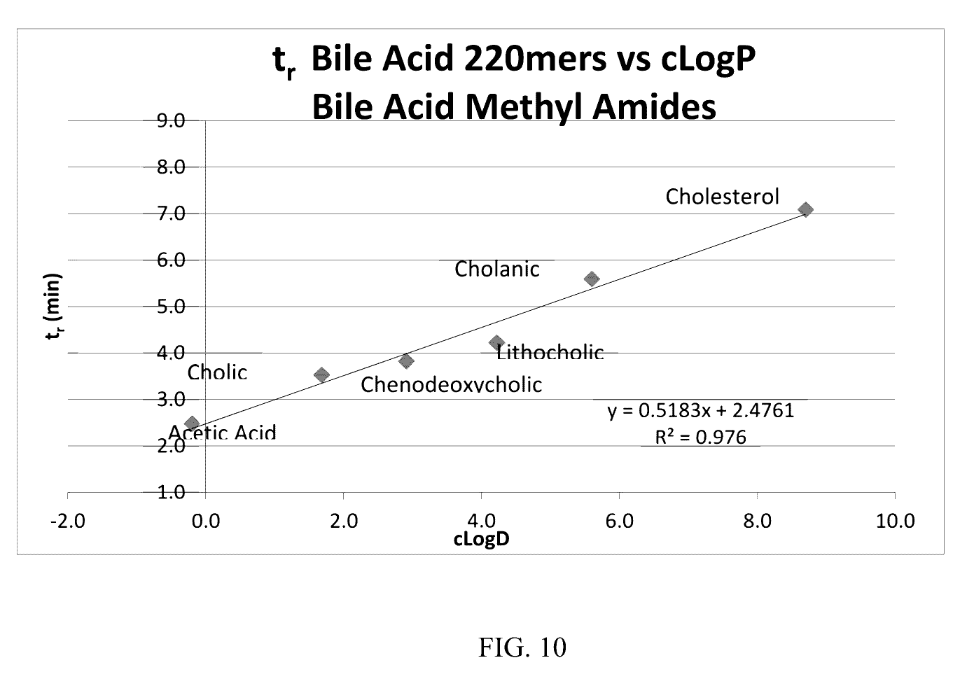

Compounds 18-24, prepared as described in Example 10 from the analogous 20 base pair oligomers were run on a reverse phase HPLC column, using an Agilent AdvanceBio Oligonucleotide column and acetonitrile/ammonium acetate gradient. Solvent A was 10 mM ammonium acetate and solvent B was acetonitrile. As illustrated in FIG. 9, the steroids were resolved by HPLC, with acetate eluting in the void volume. Accordingly, the rank order of log D can be inferred from the rank order of the retention times on a C₁₈ column. When retention times were plotted against the cLogP of acetamide (−0.2), cholic amide (1.7), chenodeoxycholic amide (2.9), lithocholic amide (4.2), cholic amide (5.6) and cholesterol acetate (8.7) a linear correlation was obtained as illustrated in FIG. 10. Without wishing to be bound by theory, inclusion of appropriate standard compounds may provide useful surrogates of absolute values of log D. It should be also noted that chromLogD and Chrom20LogD may be determined by extensions of the above experiment.

### Example 12

### Determination of cLogD and eLogD of Linear Peptides

A set of 9 linear peptides (N terminal acetylated and C terminal amide) were synthesized using standard methods and cLogD and eLogD values determined using conventional procedures. The eLogD of FFFF could not be determined because of formation of a visible aggregate at the octanol-water interface.

TABLE 1

| Sequence | cLogP | cLogD (.7.4) | eLogD (7.4) |
|---|---|---|---|
| WGG | −2.02 | −2.02 | −0.80 |
| SLI | −1.10 | −1.10 | −0.06 |
| YTGFL | −0.65 | −0.65 | 0.57 |
| ALLGF | −0.12 | −0.12 | 1.05 |
| AYL | −0.03 | −0.04 | −0.06 |
| RFL | −1.03 | −3.05 | −1.12 |
| EIF (D718) | 0.00 | −3.06 | −2.30 |
| IFA | 0.35 | 0.35 | 0.46 |
| IMF | 1.00 | 1.00 | 1.25 |
| FFFF (D721) | 3.45 | 3.45 | Not measurable |

### Example 13

### Determination of ChromLogD of Linear Peptides

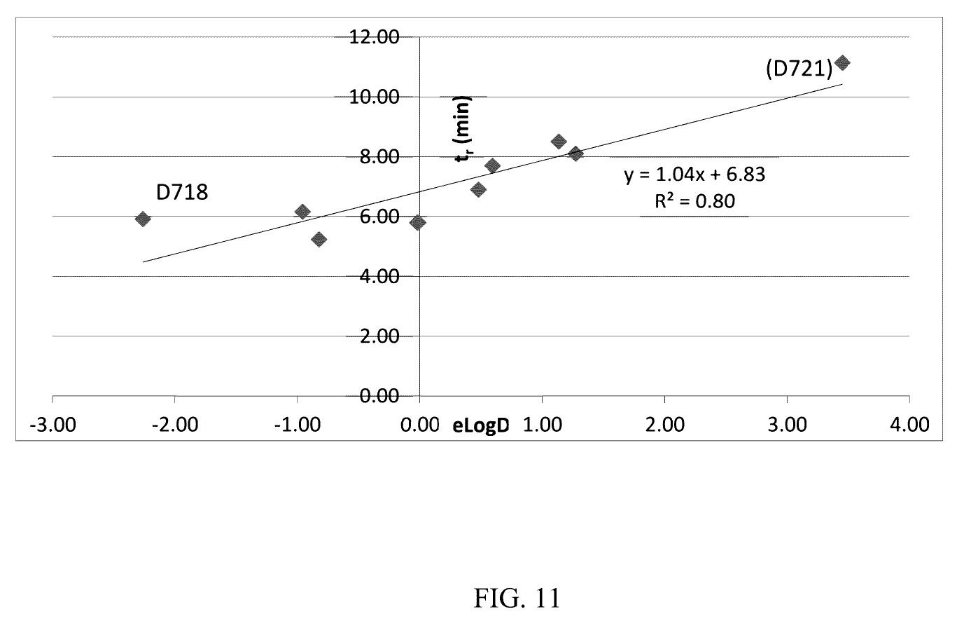

Determination of chromLogD of the peptides listed in Example 12 was performed at 25° C. using a gradient of 40 mM ammonium acetate, pH 7.4 and acetonitrile and a Water's Shield RP18 column. When retention time (min) was plotted against eLogD, a correlation coefficient of R²=0.80 was obtained (FIG. 11).

### Example 14

### General Procedure for Coupling Peptides to Extended Oligomers

40 uL of a 50% aqueous suspension of DEAE-Sepharose was added to a well in a 384-well filter plate. The resin was washed three times with 90 uL of 10 mM acetic acid in water with 0.02% Tween-20, then spun dry in a centrifuge. Purified amino-TEG modified oligomer prepared by Trilink (amino-triethyleneglycol-5′ -GCTCGTCGCATTCGGCACGC-3′) (SEQ. ID. NO. 7) (predissolved in water to 7.905 nmoles oligo per uL) 5 uL was dissolved in 785 uL of 10 mM acetic acid in water with 0.02% Tween-20 and 60 uL was loaded onto DEAE-Sepharose per well (12 wells total). The suspension was and then spun dry in a centrifuge through the filter plate into a deep well plate. The contents of the deep well plate were aspirated back on top of the resin, and then spun dry in a centrifuge again. This step was repeated once more.

The resin was washed with water (90 uL), 50% dimethylacetamide (DMA)/water (90 uL), and DMA (3×90 uL) and spun dry in a centrifuge. 50 uL 20% triethylamine in DMA was added to the well, and allowed to stand at room temperature for 7 minutes.

The resin was filtered, then washed with DMA (3×90 uL), 50% MeOH/DMA (3×90 uL) and then spun dry in a centrifuge.

In the meantime, a 200 mM solution in DMA of each of the peptide-carboxylic acid to be attached to the oligomer was prepared. 5 uL of this solution was individually aliquoted into separate wells of a fresh 384 deep well plate, and then 15 uL freshly prepared DMTMM solution (33.5 mM in 1:1 MeOH/DMA) was mixed in to each well. A 20 uL aliquot of each individual resulting solution was added to separate individual filter plate wells containing the oligomer, which was then kept at room temperature for 30 minutes.

The resin was and then spun dry in a centrifuge, rinsed with 50% MeOH/DMA (90 uL) and then spun dry in a centrifuge.

Another 5 uL of this 200 mM peptide-carboxylic acid solution was individually aliquoted into separate wells, and then 15 uL freshly prepared DMTMM solution (33.5 mM in 1:1 MeOH/DMA) was mixed in to each well. A 20 uL aliquot of each individual resulting solution was added to separate individual filter plate wells containing the oligomer, which was then kept at room temperature for 30 minutes, filtered and the resin rinsed with DMA (3×90 uL), 50% DMA/water (90 uL), and water (3×90 uL) and then spun dry in a centrifuge.

To each well was added 50 ul of 100 mM aqueous sodium hydroxide and this allowed to sit at room temperature for 10 minutes. The plate was filtered, washed with water (3×90 uL) and then spun dry in a centrifuge.

Using a new, clean, 384 deep well receiver plate, the oligomers were eluted from the well with 33 uL 1.5 M triethylammonium acetate in 5% IPA/water and allowed to stand for 4 minutes and then then spun dry in a centrifuge. This process repeated twice more so that 99 uL total was eluted through resin (3×33 uL).

### Example 15

### Chromatographic Method for Estimation of Lipophilicity of Peptides Operatively Linked to a Operatively Linked to a 20 Base Pair Oligonucleotide

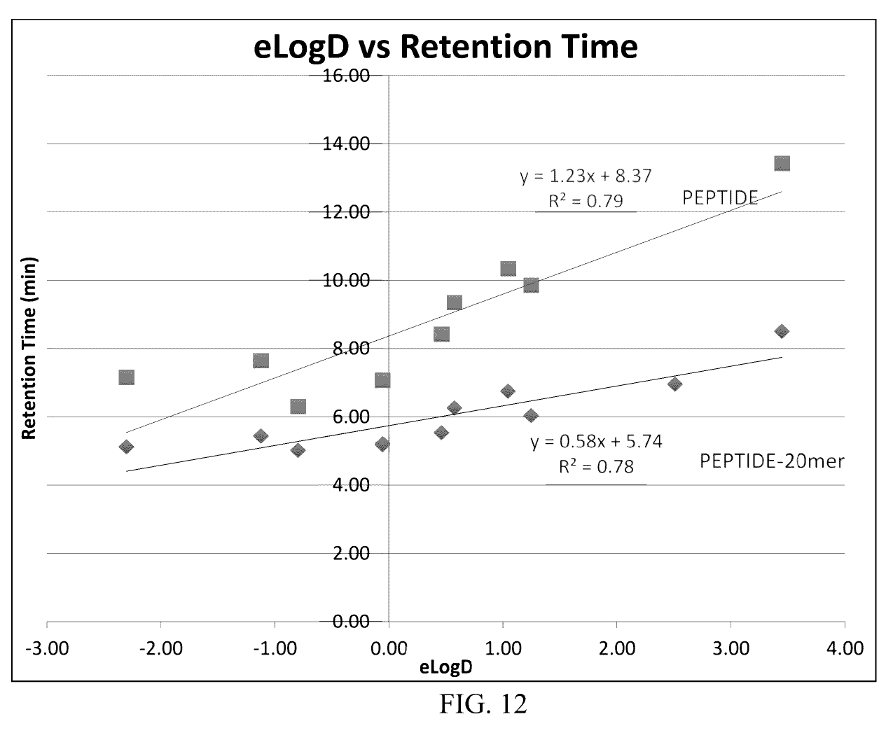

Determination of chromLogD of peptides of Example 12 linked to 20 base pair oligomers as described in Example 14 was performed at 25° C. using a gradient of 40 mM ammonium acetate, pH 7.4 and acetonitrile and a Water's Shield RP18 column. When retention time (min) was plotted against eLogD, except for peptides FFFF and FFF where cLogD was used, a correlation coefficient of R²=0.79 was obtained (FIG. 12) for the peptides linked to a 20 bp oligomer. The corresponding plot for the free peptides is also shown for the sake of comparison.

1. A method of estimating LogD of two or more compounds which include a ligand operatively linked to a recognition element comprising:
contacting the compounds with a lipid matrix;
separating compounds absorbed by the lipid matrix from compounds not absorbed by the lipid matrix; and
measuring the amounts of compounds absorbed by the lipid matrix and/or the amounts of compounds not absorbed by the lipid matrix wherein measurement of the amounts of compounds absorbed by the lipid matrix and/or the amounts of the compounds not absorbed by the lipid matrix provides an estimate of LogD of the compounds.

2. The method of claim 1, further comprising including an excipient in the contacting step, wherein the excipient is a cation, polycation or a volume excluding agent.

3. The method of claim 1, wherein the lipid matrix is a bead coated with a lipid membrane material.

4. The method of claim 3, wherein compounds absorbed by the lipid matrix are separated from compounds not absorbed by the lipid matrix by centrifugation or filtration.

5. The method of claim 3, wherein the amounts of compounds absorbed by the lipid matrix and/or the amounts of compounds not absorbed by the matrix are measured by absorbance, fluorescence, DNA sequencing or qPCR.

6. The method of claim 1, wherein two or standard compounds of measured absorbance and/or non-absorbance and measured LogD are included in the contacting step.

7. The method of claim 6, wherein comparison of the amounts of compounds absorbed by the lipid matrix and/or the amounts of compounds not absorbed by the lipid matrix with the amounts of standard compounds absorbed by the lipid matrix and/or the amounts of the standard compounds not absorbed by the lipid matrix provides an estimate of LogD of the compounds.

8. A method of estimating LogD of two or more compounds which includes a ligand operatively linked to a recognition element comprising:
contacting the compounds with a gel matrix;
applying a voltage gradient to the gel matrix; and
measuring the R_{f} of the compounds on the gel matrix wherein the R_{f} of the compounds on the gel matrix provides an estimate of LogD of the compounds.

9. The method of claim 8, wherein the gel matrix includes agarose and vesicles.

10. The method of claim 9, wherein two or more standard compounds of measured mobility on the gel matrix and measured log D are included in the contacting step.

11. The method of claim 10, wherein comparison of R_{f} of the compounds with R_{f} of the standard compounds on the gel matrix provides an estimate of LogD of the compounds.

12. The method of claim 11, further comprising isolating discrete regions of the gel matrix which include compounds.

13. The method of claim 12, wherein the compounds are eluted from each discrete region of the gel matrix to provide a unique fraction, each fraction bar coded with a unique oligonucleotide, pooled, amplified by the polymerase chain reaction and sequenced by NextGen sequencing.

14. The method of claim 13, wherein comparison of the mobility of the compounds on the gel matrix with the mobility of the standard compounds on the gel matrix provides an estimate of LogD of the compounds.

15. A method of estimating LogD of two or more compounds which include a ligand operatively linked to a recognition element comprising:
contacting the compounds with a chromatographic matrix; and
measuring the retention times of the compounds on the chromatographic matrix wherein the measured retention times of the compounds on the chromatographic matrix provides an estimate of LogD of the compounds.

16. The method of claim 15, wherein the recognition element is a single stranded DNA oligonucleotide, the ligand is a peptide or organic molecule and the chromatographic matrix is a reverse phase support.

17. The method of claim 15, wherein the recognition element is double stranded DNA, the ligand is a peptide or organic molecule and the chromatographic matrix is a reverse phase chromatographic support.

18. A compound of Formula (I):
RE-(X₁)—(C₁X₂)ₙ—(C₂X₃)ₒ-Lₐ (I)
or salts, hydrates, or solvates thereof wherein:
RE is a recognition element;
Lₐ is a ligand;
C₁ and C₂ are independently connecting elements;
n and o are independently 0 or 1; and
X₁, X₂ and X₃ are functional groups.

19. A double stranded DNA molecule comprising one oligonucleotide operatively linked to a variable first ligand at the 3′ terminus hybridized to a complementary oligonucleotide operatively linked to a constant second ligand at the adjacent 5′ terminus wherein the LogD of the second ligand is greater than 6.

20. A method of estimating LogD of two or more first ligands of double stranded DNA molecules of claim 19 comprising:
contacting the double stranded DNA molecules with a lipid matrix;
separating the double stranded DNA molecules absorbed by the lipid matrix from double stranded DNA molecules not absorbed by the lipid matrix; and
measuring the amounts of double stranded DNA molecules absorbed by the lipid matrix and/or the amounts of double stranded DNA molecules not absorbed by the matrix wherein measurement of the amounts of double stranded DNA molecules absorbed by the matrix and/or the amounts of the double stranded DNA molecules not absorbed by the matrix provide an estimate of LogD of the first ligands of the double stranded DNA molecules.
